SEQUENCE LISTING (122 sequences)

SEQ ID NO: 1 - DNA, 20 bp, Artificial sequence
  Synthetic oligonucleotide primer.
ctttaagaaggagatatacc

SEQ ID NO: 2 - DNA, 44 bp, Artificial sequence
  Synthetic oligonucleotide primer.
tgatgatgagaacccccccctttgtcgtcgtcgtctttatagtc

SEQ ID NO: 3 - DNA, 29 bp, Artificial sequence
  Synthetic oligonucleotide primer.
ttgctaaaacttagattcttactcattgc

SEQ ID NO: 4 - DNA, 26 bp, Artificial sequence
  Synthetic oligonucleotide primer.
gccctgatatggttattcatcacagc

SEQ ID NO: 5 - DNA, 26 bp, Artificial sequence
  Synthetic oligonucleotide primer.
atgaattacagagaattgtttacagg

SEQ ID NO: 6 - DNA, 28 bp, Artificial sequence
  Synthetic oligonucleotide primer.
cttttgccagattctatgaactctatgg

SEQ ID NO: 7 - DNA, 22 bp, Artificial sequence
  Synthetic oligonucleotide primer.
aagcttgagcttgctagcatac

SEQ ID NO: 8 - DNA, 24 bp, Artificial sequence
  Synthetic oligonucleotide primer.
atgacagaagggagaaagccgcac

SEQ ID NO: 9 - DNA, 21 bp, Artificial sequence
  Synthetic oligonucleotide primer.
cacgcttcagagggaatcatg

SEQ ID NO: 10 - DNA, 27 bp, Artificial sequence
  Synthetic oligonucleotide primer.
tgtgttatgtcagcagagtatgtgtcc

SEQ ID NO: 11 - DNA, 22 bp, Artificial sequence
  Synthetic oligonucleotide primer.
gtggcgtgtttgatgtatgagg

SEQ ID NO: 12 - DNA, 24 bp, Artificial sequence
  Synthetic oligonucleotide primer.
gtgctctgtggtaatctagcgttc

SEQ ID NO: 13 - DNA, 28 bp, Artificial sequence
  Synthetic oligonucleotide primer.
atgtgttggaattataggttttttatgc

SEQ ID NO: 14 - DNA, 23 bp, Artificial sequence
  Synthetic oligonucleotide primer.
gggataatgtacgctacaactgc

SEQ ID NO: 15 - DNA, 19 bp, Artificial sequence
  Synthetic oligonucleotide primer.
ttggctgtgctcttagtgc

SEQ ID NO: 16 - DNA, 26 bp, Artificial sequence
  Synthetic oligonucleotide primer.
atgacgagagtttctttttccactgc

SEQ ID NO: 17 - DNA, 25 bp, Artificial sequence
  Synthetic oligonucleotide primer.
atggtacataaaggttctctggtgg

SEQ ID NO: 18 - DNA, 21 bp, Artificial sequence
  Synthetic oligonucleotide primer.
atgcgggtcctgctggcgttc

SEQ ID NO: 19 - DNA, 22 bp, Artificial sequence
  Synthetic oligonucleotide primer.
atgctgcatcgttggttagctc

SEQ ID NO: 20 - DNA, 24 bp, Artificial sequence
  Synthetic oligonucleotide primer.
tgattgtgacatatggcactgtgg

SEQ ID NO: 21 - DNA, 25 bp, Artificial sequence
  Synthetic oligonucleotide primer.
gtggtagatcggaaaattggtatgc

SEQ ID NO: 22 - DNA, 18 bp, Artificial sequence
  Synthetic oligonucleotide primer.
ggaagggtggtgcagcag

SEQ ID NO: 23 - DNA, 22 bp, Artificial sequence
  Synthetic oligonucleotide primer.
atgtgctatggtactcgcatcg

SEQ ID NO: 24 - DNA, 21 bp, Artificial sequence
  Synthetic oligonucleotide primer.
gtgacgcatataccttggctc

SEQ ID NO: 25 - DNA, 21 bp, Artificial sequence
  Synthetic oligonucleotide primer.
ttgtcaatttcaaaactgggc

SEQ ID NO: 26 - DNA, 21 bp, Artificial sequence
  Synthetic oligonucleotide primer.
atgtttcgagggagaacggtg

SEQ ID NO: 27 - DNA, 21 bp, Artificial sequence
  Synthetic oligonucleotide primer.
gtgtcggttatgtccttcaag

SEQ ID NO: 28 - DNA, 25 bp, Artificial sequence
  Synthetic oligonucleotide primer.
atgagatacgtccttgttttcgttc

SEQ ID NO: 29 - DNA, 20 bp, Artificial sequence
  Synthetic oligonucleotide primer.
cgggtatttagaatgcgcag

SEQ ID NO: 30 - DNA, 22 bp, Artificial sequence
  Synthetic oligonucleotide primer.
ttgcacacactaccctcttctg

SEQ ID NO: 31 - DNA, 22 bp, Artificial sequence
  Synthetic oligonucleotide primer.
tggtcgggtttgtatatgagtc

SEQ ID NO: 32 - DNA, 21 bp, Artificial sequence
  Synthetic oligonucleotide primer.
gtgctgtaagtaataggaagc

SEQ ID NO: 33 - DNA, 19 bp, Artificial sequence
  Synthetic oligonucleotide primer.
aagaaggtctacggcttgg

SEQ ID NO: 34 - DNA, 22 bp, Artificial sequence
  Synthetic oligonucleotide primer.
atgattgagtctatgggggaac

SEQ ID NO: 35 - DNA, 19 bp, Artificial sequence
  Synthetic oligonucleotide primer.
gtgcctcattcgtacctcc

SEQ ID NO: 36 - DNA, 24 bp, Artificial sequence
  Synthetic oligonucleotide primer.
acacactggtctctcattatcagc

SEQ ID NO: 37 - DNA, 21 bp, Artificial sequence
  Synthetic oligonucleotide primer.
tgctggtggtggagagtttgc

SEQ ID NO: 38 - DNA, 37 bp, Artificial sequence
  Synthetic oligonucleotide primer.
gttagatcttttctgttgggtgcggttgtagctggaa

SEQ ID NO: 39 - DNA, 40 bp, Artificial sequence
  Synthetic oligonucleotide primer.
atggttaggtcttttctgttgagcgcggttgtagttggag

SEQ ID NO: 40 - DNA, 40 bp, Artificial sequence; identical to SEQ ID NO: 39
  Synthetic oligonucleotide primer.

SEQ ID NO: 41 - DNA, 18 bp, Artificial sequence
  Synthetic oligonucleotide primer.
gtgagggtcgcacgcgtg

SEQ ID NO: 42 - DNA, 24 bp, Artificial sequence
  Synthetic oligonucleotide primer.
atgagctttgtaaggtttcttgcc

SEQ ID NO: 43 - DNA, 23 bp, Artificial sequence
  Synthetic oligonucleotide primer.
atgggatctatgatgagggcaac

SEQ ID NO: 44 - DNA, 20 bp, Artificial sequence
  Synthetic oligonucleotide primer.
atggttaaagcaggggcagc

SEQ ID NO: 45 - DNA, 18 bp, Artificial sequence
  Synthetic oligonucleotide primer.
gtggcgtttagcctcctg

SEQ ID NO: 46 - DNA, 27 bp, Artificial sequence
  Synthetic oligonucleotide primer.
ttgagtttagggatgtcagacgaaacc

SEQ ID NO: 47 - DNA, 26 bp, Artificial sequence
  Synthetic oligonucleotide primer.
aatttctataaaaacttgcttgcgtg

SEQ ID NO: 48 - DNA, 23 bp, Artificial sequence
  Synthetic oligonucleotide primer.
atggatgaagcacctgacactgg

SEQ ID NO: 49 - DNA, 22 bp, Artificial sequence
  Synthetic oligonucleotide primer.
atgacagaagacgacaagcaac

SEQ ID NO: 50 - DNA, 22 bp, Artificial sequence
  Synthetic oligonucleotide primer.
gtgccgaaaagatttatcgacg

SEQ ID NO: 51 - DNA, 19 bp, Artificial sequence
  Synthetic oligonucleotide primer.
ctggacatcaaactggacg

SEQ ID NO: 52 - DNA, 27 bp, Artificial sequence
  Synthetic oligonucleotide primer.
ttggttcttacccttattgaggatagc

SEQ ID NO: 53 - DNA, 19 bp, Artificial sequence
  Synthetic oligonucleotide primer.
attgcaaacccacagttcg

SEQ ID NO: 54 - DNA, 27 bp, Artificial sequence
  Synthetic oligonucleotide primer.
ccaatcaagattgcacatacagacagc

SEQ ID NO: 55 - DNA, 20 bp, Artificial sequence
  Synthetic oligonucleotide primer.
atgggaggcgcgtctagagc

SEQ ID NO: 56 - DNA, 18 bp, Artificial sequence
  Synthetic oligonucleotide primer.
caaagcccactggtggtg

SEQ ID NO: 57 - DNA, 16 bp, Artificial sequence
  Synthetic oligonucleotide primer.
caagcgatgtggatgc

SEQ ID NO: 58 - DNA, 22 bp, Artificial sequence
  Synthetic oligonucleotide primer.
aatgttactaacggatgcctgc

SEQ ID NO: 59 - DNA, 20 bp, Artificial sequence
  Synthetic oligonucleotide primer.
tcggttactttgggcagtgc

SEQ ID NO: 60 - DNA, 22 bp, Artificial sequence
  Synthetic oligonucleotide primer.
tatggctgtacacacaaccagc

SEQ ID NO: 61 - DNA, 20 bp, Artificial sequence
  Synthetic oligonucleotide primer.
gctgaacaggaatcttgctc

SEQ ID NO: 62 - DNA, 19 bp, Artificial sequence
  Synthetic oligonucleotide primer.
agcgcgttgctgcttcacg

SEQ ID NO: 63 - DNA, 28 bp, Artificial sequence
  Synthetic oligonucleotide primer.
ctttctaggagccttaggcctctttacc

SEQ ID NO: 64 - DNA, 25 bp, Artificial sequence
  Synthetic oligonucleotide primer.
ctcagttatgataccggagcctacg

SEQ ID NO: 65 - DNA, 19 bp, Artificial sequence
  Synthetic oligonucleotide primer.
gacgcttctgccgaaagac

SEQ ID NO: 66 - DNA, 18 bp, Artificial sequence
  Synthetic oligonucleotide primer.
ctgcgccacacctgctcc

SEQ ID NO: 67 - DNA, 24 bp, Artificial sequence
  Synthetic oligonucleotide primer.
gcttccgtataaactctacactgc

SEQ ID NO: 68 - DNA, 20 bp, Artificial sequence
  Synthetic oligonucleotide primer.
tgcgcagatgccaacagaag

SEQ ID NO: 69 - DNA, 26 bp, Artificial sequence
  Synthetic oligonucleotide primer.
caacatgcctacttctctgtaaaagc

SEQ ID NO: 70 - DNA, 22 bp, Artificial sequence
  Synthetic oligonucleotide primer.
tttcaacatgcctacttctctg

SEQ ID NO: 71 - DNA, 23 bp, Artificial sequence
  Synthetic oligonucleotide primer.
gtgcagatgtacttggtatttcc

SEQ ID NO: 72 - DNA, 22 bp, Artificial sequence
  Synthetic oligonucleotide primer.
gaccttcatcccagcgagcatc

SEQ ID NO: 73 - DNA, 22 bp, Artificial sequence
  Synthetic oligonucleotide primer.
atcaaacttcacgccagaaagg

SEQ ID NO: 74 - DNA, 19 bp, Artificial sequence
  Synthetic oligonucleotide primer.
gaccagcctcacgccggac

SEQ ID NO: 75 - DNA, 18 bp, Artificial sequence
  Synthetic oligonucleotide primer.
ttcaggcgcgaccactcc

SEQ ID NO: 76 - DNA, 18 bp, Artificial sequence
  Synthetic oligonucleotide primer.
ggaccccaagcatccaag

SEQ ID NO: 77 - DNA, 20 bp, Artificial sequence
  Synthetic oligonucleotide primer.
caccccaagagcagtacttg

SEQ ID NO: 78 - DNA, 20 bp, Artificial sequence
  Synthetic oligonucleotide primer.
caccccaagagcagtacttg

SEQ ID NO: 79 - DNA, 23 bp, Artificial sequence
  Synthetic oligonucleotide primer.
ttcgtaatacttcgacacaaacc

SEQ ID NO: 80 - DNA, 21 bp, Artificial sequence
  Synthetic oligonucleotide primer.
gaatccgaacctgattcctag

SEQ ID NO: 81 - DNA, 26 bp, Artificial sequence
  Synthetic oligonucleotide primer.
attaatattaaattgcccaattaagg

SEQ ID NO: 82 - DNA, 22 bp, Artificial sequence
  Synthetic oligonucleotide primer.
gaaggcaaacctaacacccaac

SEQ ID NO: 83 - DNA, 17 bp, Artificial sequence
  Synthetic oligonucleotide primer.
attgcaacgggagttgc

SEQ ID NO: 84 - DNA, 17 bp, Artificial sequence
  Synthetic oligonucleotide primer.
cgcatccgtggaaatcc

SEQ ID NO: 85 - DNA, 19 bp, Artificial sequence
  Synthetic oligonucleotide primer.
catgttgactcgcgacagg

SEQ ID NO: 86 - DNA, 21 bp, Artificial sequence
  Synthetic oligonucleotide primer.
caccatcaccaaatgcacatc

SEQ ID NO: 87 - DNA, 21 bp, Artificial sequence
  Synthetic oligonucleotide primer.
aagtaacacccttatgcctgc

SEQ ID NO: 88 - DNA, 19 bp, Artificial sequence
  Synthetic oligonucleotide primer.
ggcaaacctaacacccaac

SEQ ID NO: 89 - DNA, 22 bp, Artificial sequence
  Synthetic oligonucleotide primer.
aagtacgagtctgataccgcac

SEQ ID NO: 90 - DNA, 19 bp, Artificial sequence
  Synthetic oligonucleotide primer.
aatttgctgctgggttgag

SEQ ID NO: 91 - DNA, 21 bp, Artificial sequence
  Synthetic oligonucleotide primer.
gaatccgaacctgattcctag

SEQ ID NO: 92 - DNA, 24 bp, Artificial sequence
  Synthetic oligonucleotide primer.
gaaacttgctccaatgttaaaacc

SEQ ID NO: 93 - DNA, 21 bp, Artificial sequence
  Synthetic oligonucleotide primer.
aaactcgagcttcagccccag

SEQ ID NO: 94 - DNA, 25 bp, Artificial sequence
  Synthetic oligonucleotide primer.
cgcgagtataaacctcaaccctgcc

SEQ ID NO: 95 - DNA, 26 bp, Artificial sequence
  Synthetic oligonucleotide primer.
ccgcgagtataaacctcaaccctgct

SEQ ID NO: 96 - DNA, 25 bp, Artificial sequence
  Synthetic oligonucleotide primer.
cgcgagtataaatctcaaccccgcc

SEQ ID NO: 97 - DNA, 24 bp, Artificial sequence
  Synthetic oligonucleotide primer.
cgtgagtataaacctcaggcccac

SEQ ID NO: 98 - DNA, 21 bp, Artificial sequence
  Synthetic oligonucleotide primer.
agaaagcctcagcccagcctc

SEQ ID NO: 99 - DNA, 22 bp, Artificial sequence
  Synthetic oligonucleotide primer.
catatacctagcgcccaattcc

SEQ ID NO: 100 - DNA, 24 bp, Artificial sequence
  Synthetic oligonucleotide primer.
gaaccacctaagcccaatttctgc

SEQ ID NO: 101 - DNA, 21 bp, Artificial sequence
  Synthetic oligonucleotide primer.
gaatccgaacctgattcctag

SEQ ID NO: 102 - DNA, 20 bp, Artificial sequence
  Synthetic oligonucleotide primer.
cctacgcaccgcctccctag

SEQ ID NO: 103 - DNA, 24 bp, Artificial sequence
  Synthetic oligonucleotide primer.
gtattcactacttcgacgccactc

SEQ ID NO: 104 - DNA, 21 bp, Artificial sequence
  Synthetic oligonucleotide primer.
ctagaccaaccagaagactgc

SEQ ID NO: 105 - DNA, 22 bp, Artificial sequence
  Synthetic oligonucleotide primer.
cctagaccaaccagaagactgc

SEQ ID NO: 106 - DNA, 21 bp, Artificial sequence
  Synthetic oligonucleotide primer.
attctctatgaccgctctcca

SEQ ID NO: 107 - DNA, 24 bp, Artificial sequence
  Synthetic oligonucleotide primer.
tctatgatgattgcgatgttctgg

SEQ ID NO: 108 - DNA, 24 bp, Artificial sequence
  Synthetic oligonucleotide primer.
atacaggtctttgagcgatattcc

SEQ ID NO: 109 - DNA, 20 bp, Artificial sequence
  Synthetic oligonucleotide primer.
gtccggtacaccttgtgtgg

SEQ ID NO: 110 - DNA, 25 bp, Artificial sequence
  Synthetic oligonucleotide primer.
cgtaagctcaaacatcttgtgcagg

SEQ ID NO: 111 - DNA, 22 bp, Artificial sequence
  Synthetic oligonucleotide primer.
tccctctagtgtttcgtcgtgg

SEQ ID NO: 112 - DNA, 19 bp, Artificial sequence
  Synthetic oligonucleotide primer.
gttctgtgcggctttgtcg

SEQ ID NO: 113 - DNA, 22 bp, Artificial sequence
  Synthetic oligonucleotide primer.
aacatctggtgtgcctatggtc

SEQ ID NO: 114 - DNA, 18 bp, Artificial sequence
  Synthetic oligonucleotide primer.
gcgacctctcctggcagc

SEQ ID NO: 115 - DNA, 20 bp, Artificial sequence
  Synthetic oligonucleotide primer.
atcatggtatggctagcatg

SEQ ID NO: 116 - DNA, 23 bp, Artificial sequence
  Synthetic oligonucleotide primer.
ttgtcgtcgtcgtctttatagtc

SEQ ID NO: 117 - DNA, 23 bp, Artificial sequence
  Synthetic oligonucleotide primer.
ctttaagaaggagatataccatg

SEQ ID NO: 118 - DNA, 20 bp, Artificial sequence
  Synthetic oligonucleotide primer.
tgatgatgagaacccccccc

SEQ ID NO: 119 - DNA, 20 bp, Artificial sequence
  Synthetic oligonucleotide primer.
tcgtcgtcgtctttatagtc

SEQ ID NO: 120 - DNA, 42 bp, Artificial sequence
  Synthetic oligonucleotide primer.
cgcttaattaaacatatgacctatcatggtatggctagcatg

SEQ ID NO: 121 - DNA, 47 bp, Artificial sequence
  Synthetic oligonucleotide primer.
ttagttagttaccggatcccttatttgtcgtcgtcgtctttatagtc

SEQ ID NO: 122 - protein, 8 aa, Artificial sequence
  Amino acid sequence of exempary FLAG tag.
DYKDDDDK

(19) United States
(12) Patent Application Publication
(10) Pub. No.: US 2010/0143411 A1
(43) Pub. Date: Jun. 10, 2010

(54) METHOD FOR IDENTIFICATION OF T-LYMPHOCYTE ANTIGENS

(75) Inventors: Wendy Catherine Brown, Palouse, WA (US); Job Enoch Lopez, Hamilton, MT (US); Robert A. Heinzen, Hamilton, MT (US); Paul A. Beare, Hamilton, MT (US)

Correspondence Address: KLARQUIST SPARKMAN, LLP, 121 SW SALMON STREET, SUITE 1600, PORTLAND, OR, 97204, US

(73) Assignees: Washington State University; The Government of the U.S.A. as represented by the Secretary, Dept. of Health and Human Service

(21) Appl. No.: 12/473,096

(22) Filed: May 27, 2009

Related U.S. Application Data
(60) Provisional application No. 61/056,359, filed on May 27, 2008.

(51) Int. Cl. A61K 39/02; C12Q 1/02; C07K 14/00; C12N 9/48; C07H 21/00; A61K 39/00; A61P 31/04

(52) U.S. Cl. 424/234.1; 435/29; 530/350; 530/395; 435/212; 530/359; 536/22.1; 424/184.1

(57) ABSTRACT

A method for high-throughput identification of antigens is disclosed. The method involves generating transcriptionally active PCR (TAP) products of one or more antigen candidates and expressing the TAP products in an in vitro translation transcription (IVTT) system. The TAP products are purified using identifiable tags. The purified TAP products are presented to isolated antigen-presenting cells (APCs), which are in turn are presented to T-cells. The ability of the antigen candidates to induce activation of the T-cells is determined. Activation of the T-cells identifies the antigen candidate as an antigen. Immunogenic compositions and methods of treatment using such compositions are also disclosed.

### CROSS REFERENCE TO RELATED APPLICATION

This application claims the benefit of U.S. Provisional Application No. 61/056,359, filed May 27, 2008, which is incorporated herein by reference in its entirety.

### ACKNOWLEDGMENT OF GOVERNMENT SUPPORT

This invention was made with government support under grant No. AI-053692 awarded by the National Institutes of Health and USDA-ARS Cooperative Agreement No. 58-5348-3-212. The government has certain rights in the invention.

### FIELD

This application relates to the field of antigen identification and specifically to high-throughput identification of T-lymphocyte antigens expressed by in vitro translation and transcription and bead-affinity purification.

### BACKGROUND

The development of rapid and sensitive techniques to identify antigens from complex pathogens that stimulate T-lymphocyte responses is critical for accelerating vaccine development. Previously, proteins from parasites, such as the protozoal pathogen Babesia bovis, were fractionated by size and individual fractions tested for T-lymphocyte recognition. Over the course of several years, this strategy identified only a few novel antigens. A major drawback was that individual fractions contained multiple proteins and it was difficult to determine the antigenic peptides in this mixture. Furthermore, this approach required raising antisera against individual fractions, screening a parasite expression library, expressing and purifying the potential vaccine antigens as recombinant proteins, and testing the proteins in T-cell assays to identify the antigen of interest. These procedures are relatively tedious and very time-consuming. Thus, the need exists for methods to rapidly and efficiently identify vaccine antigen candidates.

### SUMMARY

The ability to rapidly screen a complex pathogen proteome for proteins that elicit recall T-lymphocyte responses from immune individuals would accelerate vaccine development. To meet this need, a method for high-throughput identification of antigens is disclosed. The method involves generating transcriptionally active PCR (TAP) products of antigen candidates and expressing the TAP products in an in vitro translation transcription (IVTT) system, for example an Escherichia coli-based IVTT system, to generate candidate vaccine antigens. The candidate vaccine antigens are purified using identifiable tags incorporated into the TAP product when generated. The candidate vaccine antigens are presented to isolated antigen presenting cells (APCs) (such as B-cells, dendritic cells and/or macrophages). The APCs that have been contacted with the expressed and purified TAP products are presented to T-cells, for example CD4⁺ and/or CD8⁺ T-cells. The ability of the candidate vaccine antigens to induce activation of the T-cells is determined, for example by detecting proliferation of the T-cells. Activation of the T-cells identifies the candidate vaccine antigens as an antigen. In some examples, the TAP products include a FLAG tag and/or a 6×His tag as an identifiable tag. The use of identifiable tags, such as FLAG tags and/or 6×His tags, allows for the rapid purification of the TAP products. In some embodiments of the methods disclosed herein, purifying the TAP product using the identifiable tag includes using an antibody that specifically binds the identifiable tag. In some examples, the antibody that specifically binds the identifiable tag is attached to a solid support, such as a microbead. In some examples, the TAP products include a transcription promoter (such as T7 promoter) and a transcription terminator (such as a T7 terminator). The disclosed method is equally applicable to the high-throughput analysis of multiple antigen candidates, for example multiple antigen candidates derived from the genomic sequence of one or more pathogens of interest, such as Anaplasma marginale.

Also disclosed are immunogenic compositions that include an antigen from a pathogen of interest, for example an antigen from a pathogen of interest identified using the disclosed methods. In some examples, an immunogenic composition includes one or more of outer-membrane (OMP)4, OMP1, OMP2, OMP3, OMP4, OMP8, OMP9, OpAg3, elongation factor thermo unstable (EF-Tu), Ana29, VirB2, Patatin, Amino peptidase (pepA), OMA87, appendage-associated protein (AAAP) AM879, AAAP AM880, Peptidoglycan lipoprotein, AM197, AM366-2, AM529 or AM779 polypeptide from A. marginale or an antigenic fragment thereof. Methods of treating, preventing and/or inhibiting an infection from a pathogen of interest are also disclosed. The methods include administering a disclosed immunogenic composition to a subject.

The foregoing features and advantages of this disclosure will become more apparent from the following detailed description, which proceeds with reference to the accompanying figures.

### BRIEF DESCRIPTION OF THE DRAWINGS

FIG. 1 is a graph demonstrating that recombinant VirB9 bound to protein G-beads stimulates a dose-dependent CD4⁺ T-cell response. Recombinant VirB9, or negative-control B. bovis RAP-1CT, was bound to protein G-beads via an anti-FLAG epitope mAb, and 0.04, 0.2, 1, and 5 μg/ml protein G-beads were compared with recombinant protein at 1 and 10 μg/ml in a 4-day proliferation assay using a two-week polyclonal cell line from animal 04B91. Results are presented as the mean counts per minute (CPM) of triplicate cultures +1 standard deviation (SD). Samples were compared using one-way ANOVA and the Fisher's Least Significant Difference test. Proliferation to bead-bound VirB9 and recombinant VirB9 was significant (P<0.05) at all concentrations of antigen.

FIG. 2 is a graph demonstrating CD4⁺ T-cell responses to in vitro transcription translation (IVTT)-expressed protein bound by anti-FLAG mAb coated protein G-beads. VirB9 and OMP7, OMP8 and OMP9 were exresssed by IVTT to contain a FLAG epitope, bound to protein G-beads coupled with anti-FLAG mAb, and assayed with polyclonal bovine T-cell lines from outer-membrane vaccinated animal 04B91 using beads estimated to contain protein G concentrations of 0.04 to 5 μg/ml. Results are presented as the mean CPM of triplicate cultures +1 SD. Statistical analysis was performed as for FIG. 1. All IVTT-expressed proteins induced statistically significant proliferative responses when compared to negative-control antigens (anti-FLAG coated protein G-beads with IVTT E. coli lysate and anti-FLAG coated protein-coupled G-beads) using the three highest bead concentrations. Except for OMP7, all proteins stimulated significant responses (P<0.05) at the lowest bead concentration.

FIG. 3 is a bar graph showing the lack of proliferation in response to IVTT-expressed VirB9 by T-cells from a negative-control calf. A short-term T-cell line from calf 4919 immunized with an MSP1 fusion protein was cultured with 10 μg/ml outer-membranes (OM), native MSP1 or uninfected red blood cell membranes (URBC), or 0.04-5 μg/ml protein G beads bound to IVTT-expressed VirB9 or negative-control IVTT reaction using anti FLAG mAb. Results are expressed as the mean CPM of triplicate cultures +1 SD. Statistical analysis was performed as for FIG. 1. Significant responses (P<0.05) are indicated by asterisks.

FIG. 4 is a bar graph showing the lack of proliferation in response to six IVTT-expressed proteins by T-cells from a negative-control cow sample. A one-week T-cell line from cow C15 infected with B. bovis was cultured with 1 (white bars) and 10 (black bars) μg/ml B. bovis cell membranes (CM) or uninfected red blood cells (URBC) or with 1 (white bars) and 5 (black bars) μg/ml protein G bead-bound IVTT expressed proteins amino peptidase, VirB9, OMP9, Ana29, EF-Tu, and OMP4. Results are expressed as the mean CPM of triplicate cultures +1 SD. Statistical analysis was performed as for FIG. 1. A significant response (P<0.05) is indicated by an asterisk.

FIGS. 5A and 5B are digital images of Western blots of IVTT-expressed A. marginale proteins. IVTT reaction mixtures for amino peptidase (lane 1), VirB9 (lane 2), OMP9 (lane 3), Ana29 (lane 4), EF-Tu (lane 5) and OMP4 (lane 6), that were affinity purified on anti-His mAb-coupled protein G beads (FIG. 5A) or supernatants from the affinity purification after the beads were pelleted (FIG. 5B), were subjected to SDS-PAGE and immunoblotting with anti-FLAG mAb. The molecular weight markers are indicated to the left of each blot.

FIG. 6 is a bar graph showing the comparison of CD4⁺ T-cell responses to bead-affinity purified IVTT proteins or unpurified IVTT proteins. A polyclonal T-cell line from outer-membrane immunized animal 04B91 was tested in a proliferation assay with protein G-beads to which IVTT-expressed proteins were bound via anti-His mAb (black bars). Results are presented for all antigens tested at 1 μg/ml protein G beads except for VirB10 which was tested at 5 μg/ml. In a separate assay, a cell-line was tested with 0.04 μl of the same IVTT reaction mixtures (white bars). Stimulation indices (SI) were determined by dividing the mean CPM of triplicate cultures with antigen by the mean CPM of triplicate cultures with negative-control antigen (bead-bound or unpurified IVTT-expressed OMP2 or OMP3). As positive-controls for each assay, cells were stimulated with 1 μg/ml A. marginale outer-membranes (OM-dark gray bars) and 10 μg/ml native MSP2 (n MSP2-light gray bars), and the SI were determined by dividing the mean CPM of triplicate cultures with antigen by the mean CPM of triplicate cultures with negative-control URBC membrane antigen. Statistical analysis was performed as for FIG. 1. Significant responses (P<0.05) are indicated by an asterisk.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.
SEQ ID NOs: 1-121 are the nucleic acid sequences of exemplary nucleic acid primers.
SEQ ID NO: 122 is the amino acid sequence of a FLAG tag.

### DETAILED DESCRIPTION

### I. Listing of Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “a candidate antigen” includes single or plural candidate antigens and can be considered equivalent to the phrase “at least one candidate antigen.”

As used herein, the term “comprises” means “includes.” Thus, “comprising a cell” means “including a cell” without excluding other elements.

It is further to be understood that all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

Adjuvant: A vehicle used to enhance antigenicity; such as a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules.

Administration: The introduction of a composition into a subject by a chosen route. For example, if the chosen route is oral delivery, the composition is administered by introducing the composition into the mouth of the subject.

Animal: A living multi-cellular vertebrate or invertebrate organism, a category that includes, for example, mammals, such as cattle. The term mammal includes both human and non-human mammals. Similarly, the term “subject” includes both human and veterinary subjects, such as non-human primates. Thus, administration to a subject can include administration to a human subject or a veterinary subject. Particular examples of veterinary subjects include domesticated animals (such as cats and dogs), livestock (for example, cattle, horses, pigs, sheep, and goats), laboratory animals (for example, mice, rabbits, rats, gerbils, guinea pigs, and non-human primates), as well as birds, reptiles, and fish.

Antibody: A polypeptide ligand which include a light chain and/or heavy chain immunoglobulin variable region which specifically binds an epitope of an antigen, such as an identifiable tag. The term “specifically binds” refers to, with respect to an antigen such as an identifiable tag, the preferential association of an antibody or other ligand, in whole or part, with the identifiable tag. It is recognized that a minor degree of non-specific interaction may occur between a molecule, such as a antibody, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a target polypeptide as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antigen: A compound, composition, or substance that can stimulate the production of antibodies (such as functional antibodies) or a T-cell response in a mammal, including compositions that are injected, absorbed or otherwise introduced into a mammal. Examples include, but are not limited to, peptides, lipids, polysaccharides, and nucleic acids containing antigenic determinants, such as those recognized by an immune cell. In some examples, antigens include peptides derived from a pathogen of interest.

Amplification: Amplification of a nucleic acid molecule (such as a DNA or RNA molecule) refers to use of a technique that increases the number of copies of a nucleic acid molecule in a sample.

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid molecule. This cycle can be repeated multiple times.

The product of amplification can be characterized by such techniques as electrophoresis, restriction endonuclease cleavage patterns, oligonucleotide hybridization, and/or nucleic acid sequencing.

Other examples of in vitro amplification techniques include quantitative real-time PCR; reverse transcriptase PCR; real-time reverse transcriptase PCR; nested PCR; transcription-free isothermal amplification (see U.S. Pat. No. 6,033,881), repair chain reaction amplification (see PCT publication No. WO 90/01069); ligase chain reaction amplification (see published European Patent No. EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Bacterial pathogen: A bacteria that causes disease (pathogenic bacteria). Examples of pathogenic bacteria that can be used or treated in accordance with the disclosed methods include, without limitation, any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginal,e Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. cColi and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Cluster of Differentiation 4 (CD4): A T-cell surface protein that mediates interaction with the MHC class II molecule. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985).

Cluster of differentiation 8 (CD8): A transmembrane glycoprotein that serves as a co-receptor for the T-cell receptor (TCR) Like the TCR, CD8 binds to a major histocompatibility complex (MHC) molecule, but is specific for the class I MHC protein. There are two isoforms of the protein, alpha and beta, each encoded by a different gene. In humans, both genes are located on chromosome 2 in position 2p12.

The CD8 co-receptor is predominantly expressed on the surface of cytotoxic T-cells, but can also be found on natural killer cells. In one embodiment, a CD8⁺ T-cells is a cytotoxic T-lymphocytes. In another embodiment, a CD8 cell is a suppressor T-cell. To function, CD8 forms a dimer, consisting of a pair of CD8 chains. The extracellular IgV-like domain of CD8-α interacts with to the α₃ portion of the Class I MHC molecule. This affinity keeps the T-cell receptor of the cytotoxic T-cell and the target cell bound closely together during antigen-specific activation.

Cell: A plant, animal, insect, bacterial, parasite or fungal cell.

Complementary: A double-stranded DNA or RNA strand consists of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

Contacting: Placement in direct physical association, including both in solid and in liquid form.

Epitope: An antigenic determinant. These are particular chemical groups or peptide sequences on a molecule that are antigenic, such that they elicit a specific immune response. An antibody binds a particular antigenic epitope, such as an epitope of a pathogen of interest.

Expression: Translation of a nucleic acid into a protein. Proteins may be expressed and remain intracellular, become a component of the cell surface membrane, or be secreted into the extracellular matrix or medium. In some examples, expression can occur in cell free system, such as an in vitro translation transcription system.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as μL of bacteriophage lambda, plac, ptrp, ptac (ptrp-lac hybrid promoter), the T7 promoter and the like may be used.

Fungal pathogen: A fungus that causes disease. Examples of fungal pathogens that can be used in accordance with the disclosed methods and treated with the disclosed compositions include, without limitation, Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum) among others.

Heterologous: With reference to a molecule, such as an open reading frame, “heterologous” refers to molecules that are not normally associated with each other, for example as a single molecule.

Hybridization: The ability of complementary single-stranded DNA or RNA to form a duplex molecule, which also can be referred to as a hybridization complex. Nucleic acid hybridization techniques can be used to form hybridization complexes between a probe or primer and a nucleic acid molecule, such as hybridization between a primer an open reading frame of a pathogen of interest.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 50% identity)

Hybridization: 6×SSC at RT to 55° C. for 16-20 hours

Wash at least twice: 2×-3×SSC at RT to 55° C. for 20-30 minutes each.

Identifiable tag: A heterologous amino acid sequence that can be incorporated into a protein or peptide sequence that allows for the identification and/or purification of the protein or peptide. Non-limiting examples of identifiable tags that can be used in the disclosed methods include without limitation, HA tags, His tags (for example five or six histidine residues), GST tags and FLAG tags.

Immune response: A response of a cell of the immune system, such as a B-cell, T-cell, or monocyte, to a stimulus. In one embodiment, the response is specific for a particular antigen (an “antigen-specific response”). In one embodiment, an immune response is a T-cell response, such as a CD4⁺ response or a CD8⁺ response. In another embodiment, the response is a B-cell response, and results in the production of specific antibodies.

Immunogenic peptide: A peptide which comprises an allele-specific motif or other sequence, such as an N-terminal repeat, such that the peptide will bind an MHC molecule and induce a cytotoxic T-lymphocyte (“CTL”) response, or a B-cell response (for example antibody production) against the antigen from which the immunogenic peptide is derived.

In some embodiments, an immunogenic peptide is identified using the methods disclosed herein. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide. In specific non-limiting examples, an immunogenic polypeptide includes a region or fragment of OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from Anaplasma marginale.

Immunogenic composition: A composition comprising an immunogenic peptide that induces a measurable CTL response against virus expressing the immunogenic peptide, or induces a measurable B-cell response (such as production of antibodies) against the immunogenic peptide. It further refers to isolated nucleic acids encoding an immunogenic peptide, such as a nucleic acid that can be used to express the immunogenic peptide (and thus be used to elicit an immune response against this polypeptide).

For in vitro use, an immunogenic composition may consist of the isolated protein, peptide epitope, or nucleic acid encoding the protein, or peptide epitope. For in vivo use, the immunogenic composition will typically comprise the protein or immunogenic peptide in pharmaceutically acceptable carriers, and/or other agents. Any particular peptide, or nucleic acid encoding the polypeptide, can be readily tested for its ability to induce a CTL or B-cell response by art-recognized assays. Immunogenic compositions can include adjuvants, which are well known to one of skill in the art.

Immunologically reactive conditions: Includes reference to conditions which allow an antibody raised against a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. The immunologically reactive conditions employed in the methods are “physiological conditions” which include reference to conditions (such as temperature, osmolarity, pH) that are typical inside a living mammal or a mammalian cell. While it is recognized that some organs are subject to extreme conditions, the intra-organismal and intracellular environment is normally about pH 7 (such as from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

Inhibiting or treating a disease: Inhibiting the full development of a disease or condition, for example, in a subject who is at risk for a disease. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a protein, for example a peptide or nucleic acid) has been substantially separated or purified away from other biological components in which the component naturally occurs, such as other chromosomal and extrachromosomal DNA, RNA, and proteins. Proteins or peptides that have been “isolated” include proteins purified by standard purification methods. The term also embraces proteins or peptides prepared by recombinant expression in an in vitro translation transcription reaction as well as chemically synthesized proteins or peptides. Isolated does not require absolute purity, and can include protein or peptide molecules that are at least 50% isolated, such as at least 75%, 80%, 90%, 95%, 98%, 99%, or even 100% isolated.

Nucleic acid or nucleic acid molecule: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. Such polynucleotides can be synthesized, for example, using an automated DNA synthesizer or in some alternatives isolated from an organism, such as a pathogen of interest.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence.

Nucleotide: The fundamental unit of nucleic acid molecules. A nucleotide includes a nitrogen-containing base attached to a pentose monosaccharide with one, two, or three phosphate groups attached by ester linkages to the saccharide moiety.

The major nucleotides are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T) and uridine 5′-triphosphate (UTP or U).

Many modified nucleotides (nucleotide analogs) are known and can be used in oligonucleotides, such as the probes and primers for use in the disclosed methods. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

Open Reading Frame (ORF): A series of nucleotide triplets (codons) coding for amino acids without any internal termination codons. These sequences are usually translatable into a peptide, for example an ORF from the genome of A. marginale can be translatable into an A. marginale peptide. The term encompasses allelic variants and single nucleotide polymorphisms (SNPs).

Parasite: An organism that lives inside humans or other organisms acting as hosts (for the parasite). Parasites are dependent on their hosts for at least part of their life cycle. Parasites are harmful because they consume needed food, eat away body tissues and cells, and eliminate toxic waste. Examples of parasite for use with the methods and compositions disclosed herein include, without limitation, Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis), Coccidiosis (Eimeria species), Babesia (veterinary pathogens Babesia bovis, B. bigemina, B. divergens, B. caballi, B. equi, B. gibsoni, and human pathogens B. microti), Theileria (bovine pathogens Theileria annulata and T. parva) and ruminant parasites halminths, Hemonchus contortus and Ostertagia ostertagii.

Peptide: Any compound composed of amino acids, amino acid analogs, chemically bound together. Peptide as used herein includes oligomers of amino acids, amino acid analog, or small and large peptides, including polypeptides or proteins. Any chain of amino acids, regardless of length or post-translational modification (such as glycosylation or phosphorylation). “Peptide” applies to amino acid polymers to naturally occurring amino acid polymers and non-naturally occurring amino acid polymer as well as in which one or more amino acid residue is a non-natural amino acid, for example an artificial chemical mimetic of a corresponding naturally occurring amino acid. In some embodiments, a peptide from pathogenic organism, for example and antigenic peptide from an pathogenic organism. A “residue” refers to an amino acid or amino acid mimetic incorporated in a polypeptide by an amide bond or amide bond mimetic. A peptide has an amino terminal (N-terminal) end and a carboxy terminal (C-terminal) end. “Peptide” is used interchangeably with polypeptide or protein, and is used interchangeably herein to refer to a polymer of amino acid residues.

Pharmaceutically acceptable carriers or excipients: The pharmaceutically acceptable carriers or excipients of use are conventional. Remington's Pharmaceutical Sciences, by E. W. Martin, Mack Publishing Co., Easton, Pa., 15th Edition, 1975, describes compositions and formulations suitable for pharmaceutical delivery of the fusion proteins herein disclosed.

In general, the nature of the carrier or excipient will depend on the particular mode of administration being employed. For instance, for solid compositions (such as pill or tablet), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polymerizing agent: A compound capable of reacting monomer molecules (such as nucleotides) together in a chemical reaction to form covalently linked linear chains or a three-dimensional network of polymer chains. A particular example of a polymerizing agent is polymerase, an enzyme which catalyzes the 5′ to 3′ elongation of a primer strand complementary to a nucleic acid template. Examples of polymerases that can be used to amplify a nucleic acid molecule according to the disclosed methods include, but are not limited to the E. coli DNA polymerase I, specifically the Klenow fragment which has 3′ to 5′ exonuclease activity (Jacobsen et al., Eur. J. Biochem. 45:623-627, 1974), Taq polymerase, reverse transcriptase (such as HIV-1 RT), E. coli RNA polymerase, bacteriophage φ29 DNA polymerase (φ29 DNA polymerase is a processive DNA polymerase isolated from the bacteriophage φ29 and is described in for example in U.S. Pat. Nos. 5,198,543 and 5,001,050), Bst large fragment DNA polymerase (Exo(-) Bst; Aliotta et al., Genet. Anal. (Netherlands) 12:185-195, 1996) and exo(-)Bca DNA polymerase (Walker and Linn, Clinical Chemistry 42:1604-1608, 1996), phage M2 DNA polymerase (Matsumoto et al., Gene 84:247, 1989), phage φ PRD1 DNA polymerase (Jung et al., Proc. Natl. Acad. Sci. USA 84:8287, 1987), exo(-)VENT™ DNA polymerase (Kong et al., J. Biol. Chem. 268:1965-1975, 1993), T5 DNA polymerase (Chatterjee et al., Gene 97:13-19, 1991), SEQUENASE™ (U.S. Biochemicals), PRD1 DNA polymerase (Zhu and Ito, Biochim. Biophys. Acta. 1219:267-276, 1994)), T7 DNA polymerase, and T4 DNA polymerase holoenzyme (Kaboord and Benkovic, Curr. Biol. 5:149-157, 1995).

Primers: Short nucleic acid molecules, such as a DNA oligonucleotide, for example sequences of at least five nucleotides, which can be annealed to a complementary nucleic acid molecule, such as an open reading frame of an pathogen of interest, by nucleic acid hybridization to form a hybrid between the primer and the nucleic acid strand. A primer can be extended along the nucleic acid molecule by a polymerase enzyme. Therefore, primers can be used to amplify a nucleic acid molecule for example in an in vitro translation and transcription reaction, wherein the sequence of the primer is specific for the nucleic acid molecule, for example so that the primer will hybridize to the nucleic acid molecule under high or very high stringency hybridization conditions.

The specificity of a primer increases with its length. Thus, for example, a primer that includes 30 consecutive nucleotides will anneal to a sequence with a higher specificity than a corresponding primer of only 5 nucleotides. Thus, to obtain greater specificity, probes and primers can be selected that include at least 5, at least 6, at least 7, at least 8, at least 9, at least 10, at least 15, at least 20, at least 25, at least 30, at least 35, at least 40, at least 45, at least 50 or more consecutive nucleotides.

Primer pairs can be used for amplification of a nucleic acid sequence, for example, by PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, ©1991, Whitehead Institute for Biomedical Research, Cambridge, Ma.).

Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

Primers for use in the methods disclosed herein are generally at least 15 nucleotides in length, such as at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, or more contiguous nucleotides complementary to a target nucleic acid molecule, such as 20-60 nucleotides, 20-50 nucleotides, 20-40 nucleotides, or 20-30 nucleotides.

Purified: The term “purified” does not require absolute purity; rather, it is intended as a relative term. Thus, for example, a purified candidate vaccine antigen preparation is one in which the antigen referred to is more pure than the protein in its natural environment within a cell or within a in vitro transcription translation reaction chamber (as appropriate).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

T-cell: A white blood cell critical to the immune response. T-cells include, but are not limited to, CD4⁺ T-cells and CD8⁺ T-cells.

Therapeutically effective amount: An amount of a composition that alone, or together with an additional therapeutic agent(s) induces the desired response. A therapeutically effective amount of a composition including the active agent can be administered in a single dose, or in several doses, for example daily, during a course of treatment. However, the therapeutically effective amount can depend on the subject being treated, the severity and type of the condition being treated, and the manner of administration.

Transcriptionally Active PCR (TAP) product: A nucleic acid sequence that contains the minimal elements for transcription, for example a promoter and terminator.

Virus: A microscopic infectious organism that reproduces inside living cells. A virus consists essentially of a core of nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so. In some examples, a virus is a pathogen. Specific examples of viral pathogens which can be used in accordance with the disclosed methods and compositions include, without limitation, Arenaviruses (such as Guanarito virus, Lassa virus, Junin virus, Machupo virus and Sabia), Arteriviruses, Roniviruses, Astroviruses, Bunyaviruses (such as Crimean-Congo hemorrhagic fever virus and Hantavirus), Barnaviruses, Birnaviruses, Bornaviruses (such as Borna disease virus), Bromoviruses, Caliciviruses, Chrysoviruses, Coronaviruses (such as Coronavirus and SARS), Cystoviruses, Closteroviruses, Comoviruses, Dicistroviruses, Flaviruses (such as Yellow fever virus, West Nile virus, Hepatitis C virus, and Dengue fever virus), Filoviruses (such as Ebola virus and Marburg virus), Flexiviruses, Hepeviruses (such as Hepatitis E virus), human adenoviruses (such as human adenovirus A-F), human astroviruses, human BK polyomaviruses, human bocaviruses, human coronavirus (such as a human coronavirus HKU1, NL63, and OC43), human enteroviruses (such as human enterovirus A-D), human erythrovirus V9, human foamy viruses, human herpesviruses (such as human herpesvirus 1 (herpes simplex virus type 1), human herpesvirus 2 (herpes simplex virus type 2), human herpesvirus 3 (Varicella zoster virus), human herpesvirus 4 type 1 (Epstein-Barr virus type 1), human herpesvirus 4 type 2 (Epstein-Barr virus type 2), human herpesvirus 5 strain AD169, human herpesvirus 5 strain Merlin Strain, human herpesvirus 6A, human herpesvirus 6B, human herpesvirus 7, human herpesvirus 8 type M, human herpesvirus 8 type P and Human Cyotmegalovirus), human immunodeficiency viruses (HIV) (such as HIV 1 and HIV 2), human metapneumoviruses, human papillomaviruses (such as human papillomavirus-1, human papillomavirus-18, human papillomavirus-2, human papillomavirus-54, human papillomavirus-61, human papillomavirus-cand90, human papillomavirus RTRX7, human papillomavirus type 10, human papillomavirus type 101, human papillomavirus type 103, human papillomavirus type 107, human papillomavirus type 16, human papillomavirus type 24, human papillomavirus type 26, human papillomavirus type 32, human papillomavirus type 34, human papillomavirus type 4, human papillomavirus type 41, human papillomavirus type 48, human papillomavirus type 49, human papillomavirus type 5, human papillomavirus type 50, human papillomavirus type 53, human papillomavirus type 60, human papillomavirus type 63, human papillomavirus type 6b, human papillomavirus type 7, human papillomavirus type 71, human papillomavirus type 9, human papillomavirus type 92, and human papillomavirus type 96), human parainfluenza viruses (such as human parainfluenza virus 1-3), human parechoviruses, human parvoviruses (such as human parvovirus 4 and human parvovirus B 19), human respiratory syncytial viruses, human rhinoviruses (such as human rhinovirus A and human rhinovirus B), human spumaretroviruses, human T-lymphotropic viruses (such as human T-lymphotropic virus 1 and human T-lymphotropic virus 2), Human polyoma viruses, Hypoviruses, Leviviruses, Luteoviruses, Lymphocytic choriomeningitis viruses (LCM), Marnaviruses, Narnaviruses, Nidovirales, Nodaviruses, Orthomyxoviruses (such as Influenza viruses), Partitiviruses, Paramyxoviruses (such as Measles virus and Mumps virus), Picornaviruses (such as Poliovirus, the common cold virus, and Hepatitis A virus), Potyviruses, Poxviruses (such as Variola and Cowpox), Sequiviruses, Reoviruses (such as Rotavirus), Rhabdoviruses (such as Rabies virus), Rhabdoviruses (such as Vesicular stomatitis virus, Tetraviruses, Togaviruses (such as Rubella virus and Ross River virus), Tombusviruses, Totiviruses, Tymoviruses, Noroviruses, bovine herpesviruses including Bovine Herpesvirus (BHV) and malignant catarrhal fever virus (MCFV), among others.

### II Description of Several Embodiments

Disclosed herein are methods of high-throughput screening of proteins expressed by in vitro transcription and translation (IVTT) for recognition by memory CD4⁺ T-lymphocytes. As a model system to test the ability of IVTT-expressed proteins to stimulate recall T-cell responses, CD4⁺ T-lymphocytes were obtained from cattle immunized with an outer-membrane preparation of the tick-transmitted intraerythrocytic rickettsial pathogen, Anaplasma marginale. Immunization with purified outer-membranes induces significant protection against A. marginale infection and disease. Protection against infection correlates with induction of CD4⁺ T-lymphocyte and IgG2 responses, and a number outer-membrane proteins have been identified that stimulate these immune responses. Exemplary proteins include major surface protein (MSP)2, MSP3, MSP4, and type four secretion system (TFSS) proteins VirB9 and VirB10. These known T-cell antigens served as positive-controls for T-lymphocyte recognition of IVTT-expressed proteins. An additional 45 proteins were selected to express by IVTT that included proteins predicted by genome annotation to be expressed on the inner or outer-membrane, proteins of interest from 2D-electrophoretically separated outer-membranes that were recognized by immune serum IgG2 from outer-membrane vaccines, and two negative-control proteins not expressed in the outer-membrane immunogen.

An outer-membrane fraction of the rickettsial pathogen A. marginale induces protective immunity against infection and disease in cattle. Thus, as a demonstration of the viability of the methods disclosed herein, fifty selected candidate vaccine antigens identified from the A. marginale genome were expressed from TAP products using an Escherichia coli based IVTT system, and bead-affinity purified using antibodies to His and FLAG epitope tags that were placed at the ends of the TAP products. IVTT-expressed bead-bound antigens were processed and presented by antigen presenting cells (APCs) to T-lymphocytes from outer-membrane immunized animals and evaluated for immunogenicity in proliferation assays. Antigens that consistently stimulated responses were known T-cell antigens major surface protein (MSP)2, MSP3, VirB9, and VirB 10 and newly identified T-cell antigens outer-membrane protein (OMP)4, OMP9, elongation factor-Tu, Ana29, and OMA87. Specific T-cell stimulation was achieved even at low antigen concentration, and was highly sensitive when compared with unbound IVTT reaction products. Thus, the disclosed method allows rapid expression and identification of T-lymphocyte antigens for any pathogen for which the genome sequence is available.

Disclosed herein is a method to generate and express TAP products encoding FLAG and 6×His epitope-tagged vaccine candidates, such as A. marginale vaccine candidates, using an Escherichia coli-based IVTT system. This method is novel in its applicability for T-cell assays. Advantages of this technique are rapid protein expression, bead-affinity purification of the protein and consequent removal of inhibitory E. coli proteins, and rapid screening of vaccine candidate antigens with T-lymphocytes. This method identified four previously known and five newly described A. marginale T-cell antigens that stimulated significant T-lymphocyte proliferation from outer-membrane immunized animals that express distinct MHC class II haplotypes. This strategy is broadly applicable for high-throughput screening of any pathogen proteome using CD4⁺ T-lymphocytes from immune individuals. Under standard conditions, IVTT-based protein expression and T-cell proliferation assays can each be performed within a week, so within a few weeks a panel of novel immunostimulatory antigens can be identified from any pathogen for which a genome sequence is available.

A. High-Throughput Identification of T-Cell Antigens Expressed using IVTT

Disclosed is an in vitro method for high-throughput identification of antigens from a pathogen of interest, such as a bacterial, viral, fungal or parasitic pathogen, for example one or more of the pathogens listed in the foregoing listing of terms. In a specific example, a pathogen of interest is A. marginale.

The disclosed methods include generating a TAP product of an open reading frame (ORF) of an antigen candidate of a pathogen of interest. The generated the TAP product includes an identifiable tag, transcription promoter and a transcription terminator. The TAP product is expressed in an IVTT system to generate a candidate vaccine antigen. In some embodiments, the IVTT system is an Escherichia coli-based IVTT system, such as a cell-free expression machine, for example, the RTS (Rapid Translation System) 100 commercially available from Roche. In some examples, TAP products are generated for multiple antigen candidates, for example from multiple ORFs present in the genome of a pathogen of interest. In some examples, the TAP products of multiple antigen candidates are generated from a genome sequence A. marginale.

The candidate vaccine antigen is purified using the identifiable tag, for example to remove any proteins or other components of the IVTT system that may interfere with subsequent analysis of the candidate vaccine antigen. The purified candidate vaccine antigen is presented to isolated APCs, such as B-cells, dendritic cells, macrophages or a combination thereof, for example to load the APC with candidate vaccine antigen. In some examples, APCs are obtained from a subject that has not been immunized with one or more antigens from a pathogen of interest. The APCs are contacted with isolated T-cells and the T-cells are assayed for antigen-induced activation of the T-cells, wherein activation of the T-cells identifies the candidate vaccine antigen as an antigen. Several non-limiting examples of measuring antigen-induced activation of the T-cells include the measurement of cytokine production from the T-cells, for example by ELISA or ELISPOT assay, measurement of T-cell activation markers by flow cytometry or induced T-cell proliferation. In some examples, activation of T-cells is assayed by determining the proliferation of the T-cells. In some examples, the T-cells are CD4⁺ T-cells, CD8⁺ cells or a combination of CD4⁺ cells and CD8⁺ cells. In some examples, the T-cells are obtained from a subject previous immunized with one or more antigens from the pathogen of interest.

In some embodiments, generating a TAP product includes contacting the open reading frame of an antigen candidate with a first oligonucleotide primer and second oligonucleotide primer, wherein both the first primer and the second primer are capable of hybridizing to and amplifying the open reading frame. In some embodiments, the open reading frame is amplified, for example by PCR, to generate a first amplification product. In some examples, the first oligonucleotide primer includes a nucleotide sequence capable of hybridizing to the open reading frame, a first nucleic acid sequence heterologous to the open reading frame and optionally a nucleic acid sequence encoding an identifiable tag. In some examples, the second oligonucleotide primer includes a nucleotide sequence capable of hybridizing to the open reading frame, a second nucleic acid sequence heterologous to the open reading frame and optionally a nucleic acid sequence encoding an identifiable tag. In some embodiments, the first and second nucleic acid sequences heterologous to the open reading frame are not identical and/or complementary. By including first and second nucleic acid sequences that are heterologous to the open reading frame, when the open reading frame is amplified the resulting amplification product has heterologous nucleic acid sequences introduced at the 5′ and 3′ ends. These heterologous sequences can serve as sites of hybridization for additional primers in second, third, fourth or more rounds of PCR, for example to introduce additional identifiable tags and/or transcription promoters and/or terminators to the ends of the first amplification product.

In some embodiments, the first amplification product is contacted with a third oligonucleotide primer and fourth oligonucleotide primer, both of which are capable of hybridizing to and amplifying the first amplification product. In some examples, the third oligonucleotide primer includes a nucleic acid sequence encoding a transcription promoter, a nucleic acid sequence capable of hybridizing to the first or second heterologous nucleic acid sequence if present in the first amplification product and optionally a nucleic acid sequence encoding an identifiable tag. In some examples, the fourth oligonucleotide primer includes a nucleic acid sequence encoding a transcription terminator, a nucleic acid sequence capable of hybridizing to the first or second heterologous nucleic acid sequence if present in the first amplification product and optionally a nucleic acid sequence encoding an identifiable tag. In some examples, the first amplification product is amplified with the third and fourth primers, for example using PCR, to generate a TAP product that includes an identifiable tag, a transcription promoter, for example a T7 promotor, and a transcription terminator, for example a T7 terminator.

Examples of identifiable tags that can be used in the disclosed methods include without limitation, HA tags, His tags (for example five or six histidine residues), GST tags, FLAG tags, and the like. In some embodiments, the identifiable includes a FLAG tag, a 6×His tag, or both a FLAG tag and a 6×His tag.

In some embodiments, the candidate vaccine antigen is purified with an antibody that specifically binds the identifiable tag, for example by contacting the candidate vaccine antigen with an antibody that specifically binds the identifiable tag. Antibodies that specifically bind protein tags, such as FLAG tags, 6×His tags and HA tags are readily available and known to those of ordinary skill in the art. In some examples, the antibody that specifically binds an identifiable tag is attached to a solid support, such as one or more microbeads. Antibodies can be attached to a solid support using a variety of methods known in the art, for example adsorption, covalent linkage or streptavidin:biotin interaction among others. In some examples, the candidate vaccine antigen is presented to the APCs while still bound to the antibodies that are attached to the microbeads. In other examples, the candidate vaccine antigen is presented to the APCs while still bound to the antibodies that have been removed from the microbeads. In other examples, the candidate vaccine antigen is presented to the APCs after removal of the antibodies that are attached to the microbeads.

B. Antigens

The present disclosure relates to antigens and immunogenic composition for use in immunizing a subject against a pathogen of interest, such as but not limited to a pathogen listed in the foregoing listing of terms. In a specific example, the antigen is an antigen identified with the methods disclosed herein. In some examples, the antigen is an antigen from A. marginale. In some examples, the antigen is OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale, or an immunogenic fragment thereof [the numbering (AM###) refers the annotated genome of A. marginale].

The immunogenic antigens, such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale, or immunogenic fragments disclosed herein can be chemically synthesized by standard methods, or can be produced recombinantly. An exemplary process for polypeptide production is described in Lu et al., Federation of European Biochemical Societies Letters. 429:31-35, 1998. They can also be isolated by methods including preparative chromatography and immunological separations.

In other embodiments, fusion proteins are provided including a first and second polypeptide moiety in which one of the protein moieties includes an amino acid sequence of an i OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale, or a fragment thereof. The other moiety is a heterologous protein such as a carrier protein and/or an immunogenic protein. Such fusions also are useful to evoke an immune response against the antigen of interest, such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 from A. marginale, or a fragment thereof. In certain embodiments, an immunogenic antigen of a pathogen of interest, such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 from A. marginale, or a fragment thereof, is covalently or non-covalently linked to a toll-like receptor (TLR) ligand or dendritic cell or B-cell targeting moiety.

An antigen from a pathogen of interest can be covalently linked to a carrier, which is an immunogenic macromolecule to which an antigenic molecule can be bound. When bound to a carrier, the bound polypeptide becomes more immunogenic. Carriers are chosen to increase the immunogenicity of the bound molecule and/or to elicit higher titers of antibodies against the carrier which are diagnostically, analytically, and/or therapeutically beneficial. Covalent linking of a molecule to a carrier can confer enhanced immunogenicity and T-cell dependence (see Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, polysaccharides, polypeptides or proteins from bacteria or viruses), semi-synthetic or synthetic materials containing one or more functional groups to which a reactant moiety can be attached. Bacterial products and viral proteins (such as hepatitis B surface antigen and core antigen) can also be used as carriers, as well as proteins from higher organisms such as keyhole limpet hemocyanin, horseshoe crab hemocyanin, edestin, mammalian serum albumins, and mammalian immunoglobulins. Additional bacterial products for use as carriers include bacterial wall proteins and other products (for example, streptococcal or staphylococcal cell walls and lipopolysaccharide (LPS)).

Most antigenic epitopes are relatively small in size, such as about 5 to 100 amino acids in size, for example about 5, about 6, about 7, about 8, about 9, about 10, about 15, about 20, about 25, about 30, about 40, about 50, about 60, about 70, about 80, about 90, or about 100. Thus, fragments (for example, epitopes or other antigenic fragments) of OMP4, O OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale, can be used as an immunogen.

The present disclosure concerns nucleic acid constructs including polynucleotide sequences that encode antigenic OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale, or a fragment thereof. These polynucleotides include DNA, cDNA and RNA sequences which encode the polypeptide of interest. Methods for the manipulation and insertion of the nucleic acids of this disclosure into vectors are well known in the (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

Typically, the nucleic acid constructs encoding the antigenic polypeptides of this disclosure are plasmids. However, other vectors (for example, viral vectors, phage, cosmids, etc.) can be utilized to replicate the nucleic acids. In the context of this disclosure, the nucleic acid constructs typically are expression vectors that contain a promoter sequence which facilitates the efficient transcription of the inserted genetic sequence of the host. The expression vector typically contains an origin of replication, a promoter, as well as specific nucleic acid sequences that allow phenotypic selection of the transformed cells.

More generally, polynucleotide sequences encoding the antigenic polypeptides of this disclosure can be operably linked to any promoter and/or enhancer that is capable of driving expression of the nucleic acid following introduction into a host cell. A promoter is an array of nucleic acid control sequences that directs transcription of a nucleic acid. A promoter includes necessary nucleic acid sequences (which can be) near the start site of transcription, such as in the case of a polymerase II type promoter (a TATA element). A promoter also can include distal enhancer or repressor elements which can be located as much as several thousand base pairs from the start site of transcription. Both constitutive and inducible promoters are included (see, for example, Bitter et al., Methods in Enzymology 153:516-544, 1987).

To produce such nucleic acid constructs, polynucleotide sequences encoding antigenic polypeptides are inserted into a suitable expression vector, such as a plasmid expression vector. Procedures for producing polynucleotide sequences encoding antigenic polypeptides and for manipulating them in vitro are well known to those of skill in the art, and can be found, for example in Sambrook and Ausubel, supra.

DNA sequences encoding an antigenic polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

The polynucleotide sequences encoding an antigenic polypeptide can be inserted into an expression vector including, but not limited to, a plasmid, virus or other vehicle that can be manipulated to allow insertion or incorporation of sequences and can be expressed in either prokaryotes or eukaryotes. Hosts can include microbial, yeast, insect, and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA can be carried out by conventional techniques that are well known to those of ordinary skill in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding an antigenic polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

C. Immunogenic Compositions and Therapeutic Methods

Any of the antigenic polypeptides and nucleic acid molecules encoding the OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptides can be used as immunogens, or to produce immunogens to elicit an immune response (immunogenic compositions) to A. marginale, for example to reduce A. marginale infection or a symptom of a A. marginale infection. Following administration of a therapeutically effective amount of the disclosed therapeutic compositions, the subject can be monitored for A. marginale infection, symptoms associated with A. marginale infection, or both. Disclosed herein are methods of administering the therapeutic molecules disclosed herein (such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptides and nucleic acids encoding OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptides, or fragments thereof) to reduce A. marginale. In some examples, a therapeutically effective amount of a OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide or immunogenic fragment thereof is administered to a subject, such as a bovine subject.

In certain embodiments, the immunogenic composition includes an adjuvant. In one example, the immunogenic composition is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770, all of which are incorporated by reference herein in their entirety. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, for example, Schmolka, J. Am. Oil. Chem. Soc. 54:110, 1977, and Hunter et al., J. Immunol 129:1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the antigen in oil-in-water emulsion, to provide a vehicle for the desired antigen, and preferably has a melting temperature of less than 65° C. such that an emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

Immunogenic compositions can be formulated with an appropriate solid or liquid carrier, depending upon the particular mode of administration chosen. If desired, the disclosed pharmaceutical compositions can also contain minor amounts of non-toxic auxiliary substances, such as wetting or emulsifying agents, preservatives, and pH buffering agents and the like, for example sodium acetate or sorbitan monolaurate. Excipients that can be included in the disclosed compositions include flow conditioners and lubricants, for example silicic acid, talc, stearic acid or salts thereof, such as magnesium or calcium stearate, and/or polyethylene glycol, or derivatives thereof.

Immunogenic compositions can be provided as parenteral compositions, such as for injection or infusion. Such compositions are formulated generally by mixing a disclosed therapeutic agent at the desired degree of purity, in a unit dosage injectable form (solution, suspension, or emulsion), with a pharmaceutically acceptable carrier, for example one that is non-toxic to recipients at the dosages and concentrations employed and is compatible with other ingredients of the formulation. In addition, a disclosed therapeutic agent can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions.

A form of repository or “depot” slow-release preparation can be used so that therapeutically effective amounts of the preparation are delivered into the bloodstream over many hours or days following transdermal injection or delivery. Such long-acting formulations can be administered by implantation (for example subcutaneously or intramuscularly) or by intramuscular injection. The compounds can be formulated with suitable polymeric or hydrophobic materials (for example as an emulsion in an acceptable oil) or ion-exchange resins, or as sparingly soluble derivatives, for example, as a sparingly soluble salt.

Immunogenic compositions that include a disclosed therapeutic agent can be delivered by way of a pump (see Langer, supra; Sefton, CRC Crit. Ref Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled-release systems are discussed in Langer (Science 249:1527-33, 1990).

In one example, a pump is implanted (for example see U.S. Pat. Nos. 6,436,091; 5,939,380; and 5,993,414). Implantable drug-infusion devices are used to provide patients with a constant and long-term dosage or infusion of a therapeutic agent. Such device can be categorized as either active or passive.

Active drug delivery or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, immunogenic compositions including a disclosed therapeutic agent are administered by sustained-release systems. Suitable examples of sustained-release systems include suitable polymeric materials (such as semi-permeable polymer matrices in the form of shaped articles, for example films, or mirocapsules), suitable hydrophobic materials (for example as an emulsion in an acceptable oil) or ion-exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(-)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407, exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

Immunogenic compositions can be administered for therapeutic treatments. In therapeutic applications, a therapeutically effective amount of the immunogenic composition is administered to a subject suffering from a disease, such as A. marginale infection. The immunogenic composition can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) such as by intramuscular, subcutaneous, or intravenous injection, but even oral, nasal, or anal administration is contemplated. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle (see, for example, Banga, supra). A particulate carrier based on a synthetic polymer has been shown to act as an adjuvant to enhance the immune response, in addition to providing a controlled release.

Immunogenic compositions can be formulated in unit dosage form, suitable for individual administration of precise dosages. In pulse doses, a bolus administration of an immunogenic composition that includes a disclosed antigen, such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide or a fragment thereof is provided, followed by a time-period wherein no disclosed immunogen is administered to the subject, followed by a second bolus administration. A therapeutically effective amount of an immunogenic composition can be administered in a single dose, or in multiple doses, for example daily, during a course of treatment. In specific, non-limiting examples, pulse doses of an immunogenic composition that include a disclosed antigen are administered during the course of a day, during the course of a week, or during the course of a month.

Immunogenic compositions can be administered whenever the effect (such as decreased signs, symptoms, or laboratory results of infection) is desired. Generally, the dose is sufficient to treat or ameliorate symptoms or signs of disease without producing unacceptable toxicity to the subject. Systemic or local administration can be utilized.

Amounts effective for therapeutic use can depend on the severity of the disease and the age, weight, general state of the patient, and other clinical factors. Thus, the final determination of the appropriate treatment regimen will be made by the attending clinician. Typically, dosages used in vitro can provide useful guidance in the amounts useful for in situ administration of the pharmaceutical composition, and animal models may be used to determine effective dosages for treatment of particular disorders. Various considerations are described, for example in Gilman et al., eds., Goodman and Gilman: The Pharmacological Bases of Therapeutics, 8th ed., Pergamon Press, 1990; and Remington's Pharmaceutical Sciences, 17th ed., Mack Publishing Co., Easton, Pa., 1990. Typically, the dose range for an immunogenic polypeptide is from about 0.1 tg/kg body weight to about 100 mg/kg body weight. Other suitable ranges include doses of from about 1 μg/kg to 100 mg/kg body weight. In one example, the dose is about 1.0 μg to about 50 mg, for example, 1 μg to 1 mg, such as 1 mg peptide per subject. The dosing schedule can vary from daily to as seldom as once a year, depending on clinical factors, such as the subject's sensitivity to the peptide and tempo of the subject's disease. Therefore, a subject can receive a first dose of a disclosed therapeutic molecule, and then receive a second dose (or even more doses) at some later time(s), such as at least one day later, such as at least one week later.

The pharmaceutical compositions disclosed herein can be prepared and administered in dose units. Solid dose units include tablets, capsules, transdermal delivery systems, and suppositories. The administration of a therapeutic amount can be carried out both by single administration in the form of an individual dose unit or else several smaller dose units and also by multiple administrations of subdivided doses at specific intervals. Suitable single or divided doses include, but are not limited to about 0.01, 0.1, 0.5, 1, 3, 5, 10, 15, 30, or 50 μg protein/kg/day.

The nucleic acid constructs encoding antigenic polypeptides described herein, such as OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or immunogenic fragment thereof are used, for example, in combination, as pharmaceutical compositions (medicaments) for use in therapeutic, for example, prophylactic regimens (such as vaccines) and administered to subjects (for example, bovine subjects) to elicit an immune response. For example, the immunogenic compositions described herein can be administered to a subject prior to infection with A. marginale to inhibit infection. Thus, the pharmaceutical compositions described above can be administered to a subject to elicit a protective immune response against A. marginale. To elicit an immune response, a therapeutically effective (for example, immunologically effective) amount of the nucleic acid constructs are administered to a subject, such as a bovine subject.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and QUIL A™ (saponin).

For administration of OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 nucleic acid molecules from A. marginale, the nucleic acid can be delivered intracellularly, for example by expression from an appropriate nucleic acid expression vector which is administered so that it becomes intracellular, such as by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 88:1864-8, 1991). The present disclosure includes all forms of nucleic acid delivery, including synthetic oligos, naked DNA, plasmid and viral, integrated into the genome or not.

In another approach to using nucleic acids for immunization, an immunogenic OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or immunogenic fragment thereof can also be expressed by attenuated viral hosts or vectors or bacterial vectors. Recombinant vaccinia virus, adeno-associated virus (AAV), herpes virus, retrovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one example, a viral vector is utilized. These vectors include, but are not limited to, adenovirus, herpes virus, vaccinia, or an RNA virus such as a retrovirus. In one example, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a nucleic acid sequence encoding a OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or immunogenic fragment thereof into the viral vector, along with another gene that encodes the ligand for a receptor on a specific target cell, for example, the vector is now target specific. Retroviral vectors can be made target specific by attaching, for example, a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome or attached to a viral envelope to allow target specific delivery of the retroviral vector containing the polynucleotide encoding a OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or immunogenic fragment thereof.

Suitable formulations for the nucleic acid constructs, include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the composition for use in the inventive method is formulated to protect the nucleic acid constructs from damage prior to administration. For example, the composition can be formulated to reduce loss of the adenoviral vectors on devices used to prepare, store, or administer the expression vector, such as glassware, syringes, or needles. The compositions can be formulated to decrease the light-sensitivity and/or temperature-sensitivity of the components. To this end, the composition preferably comprises a pharmaceutically acceptable liquid carrier, such as, for example, any of those described above, and a stabilizing agent selected from the group consisting of polysorbate 80, L-arginine, polyvinylpyrrolidone, trehalose, and combinations thereof.

In therapeutic applications, a therapeutically effective amount of the immunogenic composition is administered to a subject prior to or following exposure to or infection. When administered prior to exposure, the therapeutic application can be referred to as a prophylactic administration (such as in the form of a vaccine). Single or multiple administrations of the compositions are administered depending on the dosage and frequency as required and tolerated by the subject. In one embodiment, the dosage is administered once as a bolus, but in another embodiment can be applied periodically until a therapeutic result, such as a protective immune response, is achieved. Generally, the dose is sufficient to treat or ameliorate symptoms or signs of disease without producing unacceptable toxicity to the subject. Systemic or local administration can be utilized.

In the context of nucleic acid vaccines, naturally occurring or synthetic immunostimulatory compositions that bind to and stimulate receptors involved in innate immunity can be administered along with nucleic acid constructs encoding the antigenic polypeptides, such as polynucleotide encoding a OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or immunogenic fragment thereof. For example, agents that stimulate certain Toll-like receptors (such as TLR7, TLR8 and TLR9) can be administered in combination with the nucleic acid constructs encoding antigenic polypeptides. In some embodiments, the nucleic acid construct is administered in combination with immunostimulatory CpG oligonucleotides.

Nucleic acid constructs encoding a OMP4, OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale can be introduced in vivo as naked DNA plasmids. DNA vectors can be introduced into the desired host cells by methods known in the art, including but not limited to transfection, electroporation (for example, transcutaneous electroporation), microinjection, transduction, cell fusion, DEAE dextran, calcium phosphate precipitation, use of a gene gun, or use of a DNA vector transporter (See for example, Wu et al. J. Biol. Chem., 267:963-967, 1992; Wu and Wu J. Biol. Chem., 263:14621-14624, 1988; and Williams et al. Proc. Natl. Acad. Sci. USA 88:2726-2730, 1991). As described in detail in the Examples, a needleless delivery device, such as a BIOJECTOR® needleless injection device, can be utilized to introduce the therapeutic nucleic acid constructs in vivo. Receptor-mediated DNA delivery approaches can also be used (Curiel et al. Hum. Gene Ther., 3:147-154, 1992; and Wu and Wu, J. Biol. Chem., 262:4429-4432, 1987). Methods for formulating and administering naked DNA to mammalian muscle tissue are disclosed in U.S. Pat. Nos. 5,580,859 and 5,589,466. Other molecules are also useful for facilitating transfection of a nucleic acid in vivo, such as a cationic oligopeptide (for example, WO95/21931), peptides derived from DNA binding proteins (for example, WO96/25508), or a cationic polymer (for example, WO95/21931).

Another well-known method that can be used to introduce nucleic acid constructs into host cells is particle bombardment (also know as biolistic transformation). Biolistic transformation is commonly accomplished in one of several ways. One common method involves propelling inert or biologically active particles at cells. This technique is disclosed in, for example, U.S. Pat. Nos. 4,945,050, 5,036,006; and 5,100,792, all to Sanford et al. Generally, this procedure involves propelling inert or biologically active particles at the cells under conditions effective to penetrate the outer surface of the cell and to be incorporated within the interior thereof. When inert particles are utilized, the plasmid can be introduced into the cell by coating the particles with the plasmid containing the exogenous DNA. Alternatively, the target cell can be surrounded by the plasmid so that the plasmid is carried into the cell by the wake of the particle.

Alternatively, the vector can be introduced in vivo by lipofection. Synthetic cationic lipids designed to limit the difficulties and dangers encountered with liposome-mediated transfection can be used to prepare liposomes for in vivo transfection of a gene encoding a marker (Felgner et. al. Proc. Natl. Acad. Sci. USA 84:7413-7417, 1987; Mackey, et al. Proc. Natl. Acad. Sci. USA 85:8027-8031, 1988; Ulmer et al. Science 259:1745-1748, 1993). The use of cationic lipids can promote encapsulation of negatively charged nucleic acids, and also promote fusion with negatively charged cell membranes (Felgner and Ringold Science 337:387-388, 1989). Particularly useful lipid compounds and compositions for transfer of nucleic acids are described in WO95/18863 and WO96/17823, and in U.S. Pat. No. 5,459,127.

As with the immunogenic polypeptide, the nucleic acid compositions may be administered in a single dose, or multiple doses separated by a time interval can be administered to elicit an immune response. For example, two doses, or three doses, or four doses, or five doses, or six doses or more can be administered to a subject over a period of several weeks, several months or even several years, to optimize the immune response.

It will be appreciated that the exact dosage is chosen by the individual clinician in view of the subject to be treated. In general, dosage and administration are adjusted to provide an effective amount of the desired active agent to the subject being treated. As will be appreciated by those of ordinary skill in the art, the effective amount of bioactive agent may vary depending on such factors as the desired biological endpoint, the immunogenic composition and/or immunostimulatory agents to be delivered, the target tissue, the route of administration, etc. For example, the severity of the disease state; age, weight and gender of the subject being treated; diet, time and frequency of administration; drug combinations; reaction sensitivities; and tolerance/response to therapy.

The compositions can be administered in combination with various vaccines either currently being used or in development, whether intended for human or non-human subjects.

D. Kits

The immunogenic compositions disclosed herein can be provided to an administering clinician or other health care-professional in the form of a kit. The kit is a package which houses a container which contains the immunogenic composition including the vaccine and instructions for administering the composition to a subject. The kit can optionally also contain one or more other therapeutic agents and instructions for use. For example, a vaccine cocktail containing two or more vaccines can be included, or separate pharmaceutical compositions containing different vaccines or therapeutic agents. The kit can also contain separate doses of the composition for serial or sequential administration. The kit can optionally contain instructions for storage, reconstitution (if applicable), and administration of any or all therapeutic agents included. The kits can include a plurality of containers reflecting the number of administrations to be given to a subject. If the kit contains a first and second container, then a plurality of these can be present.

The subject matter of the present disclosure is further illustrated by the following non-limiting Examples.

### Examples

### Example 1

### Materials and Methods

### Animals Used in the Study

Holstein cattle numbers 04B90 and 04B91 were immunized repeatedly three years prior to the onset of study with purified A. marginale outer-membranes as described in Lopez et al., “Identification of novel antigenic proteins in a complex A. marginale outer-membrane immunogen by mass spectrometry and genomic mapping.” Infect. Immun. 73, 8109, 2005, which incorporated herein by reference in its entirety. The animals maintained strong memory CD4⁺ T-lymphocyte responses throughout the course of this study (see Lopez et al., “Immunogenicity of A. marginale type IV secretion system proteins in a protective outer-membrane vaccine.” Infect. Immun. 75, 2333, 2007). Holstein steer number 4919 was immunized with a recombinant protein containing A. marginale MSP1a T-cell epitopes and MSP1b1.

Expression of Recombinant Proteins in E. coli

Recombinant VirB9, VirB10, elongation factor-Tu (EF-Tu), and B. bovis rhoptry-associated protein-1 C-terminal region (RAP-1CT) were expressed as thioredoxin-FLAG- and 6×His-tagged fusion proteins from the pBAD/TOPO ThioFusion expression vector (INVITROGEN®, Carlsbad, Calif.) and purified by anti-FLAG affinity chromatography (Sigma-Aldrich, St. Louis, Mo.) as described in Lopez et al., Infect. Immun. 75, 2333, 2007, which is incorporated herein by reference in its entirety. Outer-membrane protein (OMP)s 2, 3, 7, 8, 9, and 12 were expressed as T7- and His-tagged fusion proteins from the pET28 vector (NOVAGEN®, Inc., EMD Biosciences, Darmstadt, Germany), and purified on nickel NTA agarose columns (QIAGEN®, Inc., Valencia, Calif.) and by anti-T7 tag affinity chromatography (NOVAGEN®) as described in Noh et al., “Differential expression and sequence conservation of the A. marginale msp2 gene superfamily outer-membrane proteins.” Infect. Immun. 74, 3471, 2006, which is incorporated herein by reference in its entirety. OMPs 10, 11 and 14 were expressed from the pBAD/TOPO THIOFUSION™ expression vector and purified by anti-thioredoxin affinity chromatography (Sigma-Aldrich) (see Noh et al., 2006).

### Amplification and Expression of Outer-Membrane Vaccine Candidate Proteins

From annotation of the A. marginale genome (see Brayton et al., “Complete genome sequencing of A. marginale reveals that the surface is skewed to two superfamilies of outer-membrane proteins.” Proc. Natl. Acad. Sci. U. S. A. 102, 844, 2005 and Brayton et al., “Genomic and proteomic approaches to vaccine candidate identification for A. marginale.” Expert Rev. Vaccines 5, 95, 2006) and previous studies that identified antigenic proteins within a protective outer-membrane immunogen (Lopez et al., 2005; Lopez et al., 2007), 50 proteins were selected as potential vaccine candidates. Genomic DNA from the St. Maries strain of A. marginale was isolated as previously described (Lopez et al., 2007). In a pilot study, A. marginale virB9, omp7, omp8, and omp9 were amplified by polymerase chain reaction (PCR) using primers for each A. marginale open reading frame (ORF) (Sigma Aldrich) and expressed by IVTT. Two rounds of PCR were performed using the Linear Generation System (LGS) from Roche (Mannheim, Germany) to produce transcriptionally active PCR (TAP) fragments, which incorporates a 6×His tag, a T7 promoter, and T7 terminator onto each A. marginale ORF. In the first round of PCR, primers for each annotated ORF (see Table 1) contained the following forward and reverse primer sequences, 5′-CTTTAAGAAGGAGATATACC-3′ (SEQ ID NO: 1) and 5′-TGATGATGAGAACCCCCCCCTTTGTCGTCGTCGTCTTTATAGTC-3′ (SEQ ID NO: 2) respectively, where the FLAG tag sequence (bold) encodes the peptide epitope DYKDDDDK (SEQ ID NO: 122) (see Table 1). A touchdown PCR amplification protocol was used that consisted of 30 cycles of 94° C. for 30 seconds, 60° C. for 45 seconds, and 72° C. for 2 minutes. This was repeated with the annealing temperature dropping −0.5° C. per cycle, a ramp of 3 degrees/second, and the gradient at 0.0. The second round of PCR incorporated LGS specific linkers containing a T7 promoter, 6×His tag and T7 terminator (Roche), and the resulting TAP fragment contained C-terminal FLAG and 6×His epitopes.

TABLE 1

Annotated A. marginale outer-membrane
proteins and primer sequences used for IVTT expression.

| Genomic | | | SEQ | | SEQ |
|---|---|---|---|---|---|
| designation | Annotation | Forward primer^{a} | ID NO: | Reverse primer^{b} | ID NO: |
| AM030 | ORFX | TTGCTAAAACTTAGAT | 3 | TCGGTTACTTTGGGC | 59 |
| | | TCTTACTCATTGC | | AGTGC | |
| AM032 | ORFY | GCCCTGATATGGTTAT | 4 | TATGGCTGTACACACA | 60 |
| | | TCATCACAGC | | ACCAGC | |
| AM090 | MSP4 | ATGAATTACAGAGAAT | 5 | GCTGAACAGGAATCTT | 61 |
| | | TGTTTACAGG | | GCTC | |
| AM127 | Hypothetical | CTTTTGCCAGATTCTA | 6 | AGCGCGTTGCTGCTTC | 62 |
| | protein | TGAACTCTATGG | | ACG | |
| AM197 | Hypothetical | AAGCTTGAGCTTGCTA | 7 | CTTTCTAGGAGCCTTA | 63 |
| | protein | GCATAC | | GGCCTCTTTACC | |
| AM254 | EF-Tu | ATGACAGAAGGGAGAA | 8 | CTCAGTTATGATACCG | 64 |
| | | AGCCGCAC | | GAGCCTACG | |
| AM529 | Hypothetical | CACGCTTCAGAGGGAA | 9 | GACGCTTCTGCCGAAA | 65 |
| | protein | TCATG | | GAC | |
| AM530 | MSP1 | TGTGTTATGTCAGCAG | 10 | CTGCGCCACACCTGCT | 66 |
| | superfamily | AGTATGTGTCC | | CC | |
| | member | | | | |
| AM532 | MSP1a-like | GTGGCGTGTTTGATGT | 11 | GCTTCCGTATAAACTC | 67 |
| | protein | ATGAGG | | TACACTGC | |
| | (MLP)2 | | | | |
| AM536 | MLP4 | GTGCTCTGTGGTAATC | 12 | TGCGCAGATGCCAACA | 68 |
| | | TAGCGTTC | | GAAG | |
| AM560 | Putative cell- | ATGTGTTGGAATTATA | 13 | CAACATGCCTACTTCT | 69 |
| | surface | GGTTTTTTATGC | | CTGTAAAAGC | |
| | protein | | | | |
| AM573 | Paralog of | GGGATAATGTACGCTA | 14 | TTTCAACATGCCTACT | 70 |
| | AM560 | CAACTGC | | TCTCTG | |
| AM590 | Lipoprotein A | TTGGCTGTGCTCTTAG | 15 | GTGCAGATGTACTTGG | 71 |
| | precursor | TGC | | TATTTCC | |
| | (rlpA) | | | | |
| AM778 | Family of | ATGACGAGAGTTTCTT | 16 | GACCTTCATCCCAGCG | 72 |
| | putative | TTTCCACTGC | | AGCATC | |
| | OMPs | | | | |
| AM779 | Family of | ATGGTACATAAAGGTT | 17 | ATCAAACTTCACGCCA | 73 |
| | putative | CTCTGGTGG | | GAAAGG | |
| | OMPs | | | | |
| AM780 | Family of | ATGCGGGTCCTGCTGG | 18 | GACCAGCCTCACGCCG | 74 |
| | putative | CGTTC | | GAC | |
| | OMPs | | | | |
| AM854 | Peptidoglycan | ATGCTGCATCGTTGGT | 19 | TTCAGGCGCGACCACT | 75 |
| | lipoprotein | TAGCTC | | CC | |
| AM878 | Anaplasma | TGATTGTGACATATGG | 20 | GGACCCCAAGCATCCA | 76 |
| | appendage- | CACTGTGG | | AG | |
| | associated | | | | |
| | protein (AAAP) | | | | |
| AM879 | AAAP | GTGGTAGATCGGAAAA | 21 | CACCCCAAGAGCAGTA | 77 |
| | | TTGGTATGC | | CTTG | |
| AM880 | AAAP | GGAAGGGTGGTGCAGC | 22 | CACCCCAAGAGCAGTA | 78 |
| | | AG | | CTTG | |
| AM956 | Amino | ATGTGCTATGGTACTC | 23 | TTCGTAATACTTCGAC | 79 |
| | peptidase | GCATCG | | ACAAACC | |
| | (pepA) | | | | |
| AM987 | OMP15 | GTGACGCATATACCTT | 24 | GAATCCGAACCTGATT | 80 |
| | | GGCTC | | CCTAG | |
| AM1024 | TolC | TTGTCAATTTCAAAAC | 25 | ATTAATATTAAATTGC | 81 |
| | | TGGGC | | CCAATTAAGG | |
| AM1063 | MSP3 | ATGTTTCGAGGGAGAA | 26 | GAAGGCAAACCTAACA | 82 |
| | | CGGTG | | CCCAAC | |
| AM1076 | Ana29 | GTGTCGGTTATGTCCT | 27 | ATTGCAACGGGAGTTG | 83 |
| | | TCAAG | | C | |
| AM1096 | OMA87 | ATGAGATACGTCCTTG | 28 | CGCATCCGTGGAAATC | 84 |
| | | TTTTCGTTC | | C | |
| AM1097 | Putative OMP | CGGGTATTTAGAATGC | 29 | CATGTTGACTCGCGAC | 85 |
| | | GCAG | | AGG | |
| AM1140 | OpAg3 | TTGCACACACTACCCT | 30 | CACCATCACCAAATGC | 86 |
| | | CTTCTG | | ACATC | |
| AM1142 | OpAg2 | TGGTCGGGTTTGTATA | 31 | AAGTAACACCCTTATG | 87 |
| | | TGAGTC | | CCTGC | |
| AM1144 | MSP2 | GTGCTGTAAGTAATAG | 32 | GGCAAACCTAACACCC | 88 |
| | | GAAGC | | AAC | |
| AM1139 | OMP1 | AAGAAGGTCTACGGCT | 33 | AAGTACGAGTCTGATA | 89 |
| | | TGG | | CCGCAC | |
| AM1156 | OMP2 | ATGATTGAGTCTATGG | 34 | AATTTGCTGCTGGGTT | 90 |
| | | GGGAAC | | GAG | |
| AM1159 | OMP3 | GTGCCTCATTCGTACC | 35 | GAATCCGAACCTGATT | 91 |
| | | TCC | | CCTAG | |
| AM1164 | OMP4 | ACACACTGGTCTCTCA | 36 | GAAACTTGCTCCAATG | 92 |
| | | TTATCAGC | | TTAAAACC | |
| AM1166 | OMP5 | TGCTGGTGGTGGAGAG | 37 | AAACTCGAGCTTCAGC | 93 |
| | | TTTGC | | CCCAG | |
| AM1220 | OMP7^{c,d} | GTTAGATCTTTTCTGT | 38 | CGCGAGTATAAACCTC | 94 |
| | | TGGGTGCGGTTGTAGC | | AACCCTGCC | |
| | | TGGAA | | | |
| AM1221 | OMP8^{c} | ATGGTTAGGTCTTTTC | 39 | CCGCGAGTATAAACCT | 95 |
| | | TGTTGAGCGCGGTTGT | | CAACCCTGCT | |
| | | AGTTGGAG | | | |
| AM1222 | OMP9^{c} | ATGGTTAGGTCTTTTC | 40 | CGCGAGTATAAATCTC | 96 |
| | | TGTTGAGCGCGGTTGT | | AACCCCGCC | |
| | | AGTTGGAG | | | |
| AM1223 | OMP10 | GTGAGGGTCGCACGCG | 41 | CGTGAGTATAAACCTC | 97 |
| | | TG | | AGGCCCAC | |
| AM1255 | OMP11 | ATGAGCTTTGTAAGGT | 42 | AGAAAGCCTCAGCCCA | 98 |
| | | TTCTTGCC | | GCCTC | |
| AM1257 | OMP12 | ATGGGATCTATGATGA | 43 | CATATACCTAGCGCCC | 99 |
| | | GGGCAAC | | AATTCC | |
| AM1258 | OMP13 | ATGGTTAAAGCAGGGG | 44 | GAACCACCTAAGCCCA | 100 |
| | | CAGC | | ATTTCTGC | |
| AM075 | OMP14 | GTGGCGTTTAGCCTCC | 45 | GAATCCGAACCTGATT | 101 |
| | | TG | | CCTAG | |
| AM1314 | VirB10 | TTGAGTTTAGGGATGT | 46 | CCTACGCACCGCCTCC | 102 |
| | | CAGACGAAACC | | CTAG | |
| AM1315 | VirB9^{c} | AATTTCTATAAAAACT | 47 | GTATTCACTACTTCGA | 103 |
| | | TGCTTGCGTG | | CGCCACTC | |
| AM133 | MSP1b partial | ATGGATGAAGCACCTG | 48 | CTAGACCAACCAGAAG | 104 |
| | gene 3 | ACACTGG | | ACTGC | |
| AM180 | MSP1b1 | ATGACAGAAGACGACA | 49 | CCTAGACCAACCAGAA | 105 |
| | | AGCAAC | | GACTGC | |
| AM387-1^{e} | Hypothetical | GTGCCGAAAAGATTTA | 50 | ATTCTCTATGACCGCT | 106 |
| | protein | TCGACG | | CTCCA | |
| AM387-2^{e} | Hypothetical | CTGGACATCAAACTGG | 51 | TCTATGATGATTGCGA | 107 |
| | protein | ACG | | TGTTCTGG | |
| AM072-1^{e} | Hypothetical | TTGGTTCTTACCCTTA | 52 | ATACAGGTCTTTGAGC | 108 |
| | protein | TTGAGGATAGC | | GATATTCC | |
| AM072-2^{e} | Hypothetical | ATTGCAAACCCACAGT | 53 | GTCCGGTACACCTTGT | 109 |
| | protein | TCG | | GTGG | |
| AM072-3^{e} | Hypothetical | CCAATCAAGATTGCAC | 54 | CGTAAGCTCAAACATC | 110 |
| | protein | ATACAGACAGC | | TTGTGCAGG | |
| AM366-1^{e} | Hypothetical | ATGGGAGGCGCGTCTA | 55 | TCCCTCTAGTGTTTCG | 111 |
| | protein | GAGC | | TCGTGG | |
| AM366-2^{e} | Hypothetical | CAAAGCCCACTGGTGG | 56 | GTTCTGTGCGGCTTTG | 112 |
| | protein | TG | | TCG | |
| AM366-3^{e} | Hypothetical | CAAGCGATGTGGATGC | 57 | AACATCTGGTGTGCCT | 113 |
| | protein | | | ATGGTC | |
| AM366-4^{e} | Hypothetical | AATGTTACTAACGGAT | 58 | GCGACCTCTCCTGGCA | 114 |
| | protein | GCCTGC | | GC | |

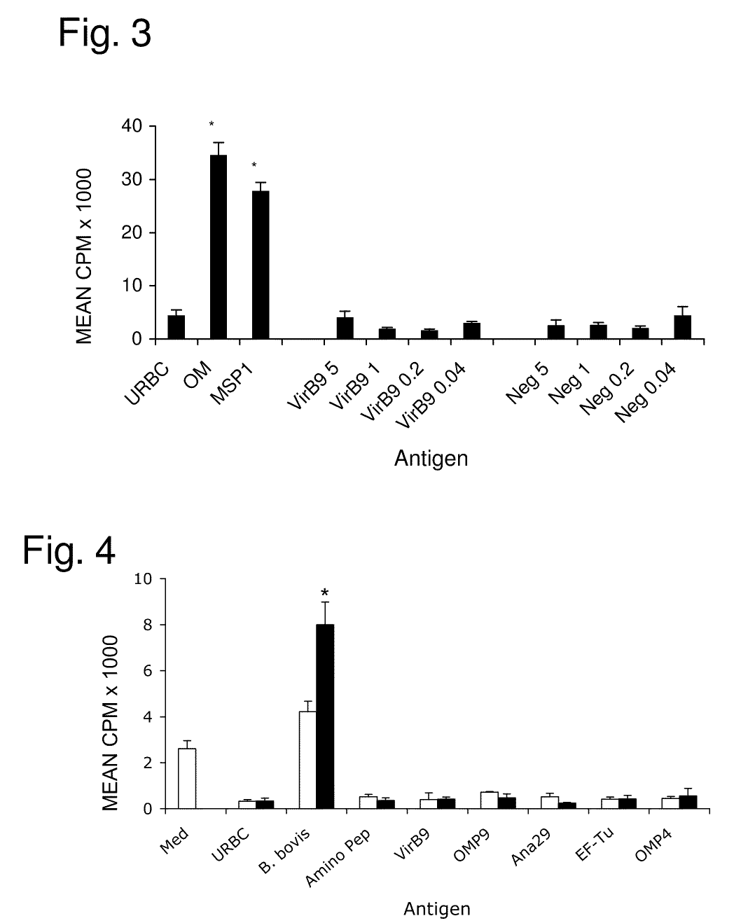

^{a}A. marginale gene-specific forward primers are shown. For screening 50 IVTT-expressed proteins, all primers also had the following sequence: ATCATGGTATGGCTAGCATG (SEQ ID NO: 115), 5′ to the A. marginale specific sequence (Table 2 and FIG. 3).
^{b}A. marginale gene-specific reverse primers are shown. For screening 50 IVTT-expressed proteins, all primers also had the following sequence: TTGTCGTCGTCGTCTTTATAGTC (SEQ ID NO: 116), 5′ to the A. marginale specific sequence (Table 2 and FIG. 3).
^{c}For amplification of VirB9, OMP7, OMP8, and OMP9 in the first IVTT expression experiment (FIG. 2), forward primers contained the following additional Roche specific sequences: CTTTAAGAAGGAGATATACCATG (SEQ ID NO: 117), 5′ to the A. marginale specific forward primer sequence, and TGATGATGAGAACCCCCCCC (SEQ ID NO: 118), 5′ to the A. marginale specific reverse primer sequence.
^{d}OMP7 was not amplified in the experiment where 50 IVTT-expressed proteins were screened (Table 2 and FIG. 3).
^{e}AM387, AM072, and AM366 encoded large ORFs, so that two or more gene fragments overlapping by at least 60 nucleotides were amplified for IVTT expression, and these fragments were designated 1-4.

In the second trial, 50 annotated ORFs (Brayton et al., 2005) were amplified by PCR, using primers synthesized by Sigma Aldrich (see Table 1), and, with a modification of the protocol described above, expressed using in vitro translation and transcription (IVTT). Three rounds of PCR were performed to produce TAP fragments for each A. marginale ORF. In the first round, forward and reverse primers for each annotated ORF contained gene-specific sequences (Table 1) and the following common sequences, 5′-ATCATGGTATGGCTAGCATG-3′ (SEQ ID NO: 115), and 5′-TCGTCGTCGTCTTTATAGTC-3′ (SEQ ID NO: 119) respectively, whereby the reverse primer contains a sequence that encodes a portion of the FLAG peptide epitope (Table 1). The touchdown amplification protocol described above was used. A second round of PCR was performed using forward (5′-CGCTTAATTAAACATATGACCTATCATGGTATGGCTAGCATG-3′, SEQ ID NO: 120) and reverse (5′-TTAGTTAGTTACCGGATCCCTTATTTGTCGTCGTCGTCTTTATAGTC-3′, SEQ ID NO: 121) primers containing linkers required for integration into the LGS. The reverse primer also allowed for the completion of the FLAG epitope—DYKDDDDK (SEQ ID NO: 122) (bold). The final PCR, resulted in the production of TAP fragments containing a T7 promoter, N-terminal 6×His sequence, T7 terminator and C-terminal FLAG epitope.

All PCR products were then purified using the High Pure UF cleanup kit (Roche) and used directly in the Rapid Translation System (RTS) 100 E. coli HY kit (Roche). All IVTT reaction mixtures were stored at −20° C.

### Dot-Blot Analysis

To verify protein expression and to estimate the amount of IVTT protein expressed, dot blots were performed. Recombinant A. marginale VirB9 thioredoxin-fusion protein that contained 6×His and FLAG epitopes (Lopez et al., 2007) was used to create a standard curve. Two μg of VirB9 thioredoxin-FLAG fusion protein were diluted two-fold to 0.015 μg in 200 μl of 1× phosphate buffered saline, pH 7.2 (PBS) and 1 μl of IVTT expressed protein was diluted in 100 μl of PBS. Samples were spotted on a pre-wetted nitrocellulose membrane using a Hybrid Dot Manifold (BRL Life Technologies, Inc., Gaithersburg, Md.). The nitrocellulose membrane was then rinsed with 200 μl of PBS, incubated for 1 hour with horseradish peroxidase-labeled (HRP) anti-penta His monoclonal antibody (mAb) (QIAGEN®) diluted at 1:3,000 in I-Block reagent (Applied Biosystems, Bedford, Ma.), and rinsed for 1 hour with I-Block reagent. Antibody binding was detected with the ECL western blotting reagent (Pierce, Rockford, Ill.). Blots were exposed for different time points ranging from 5 seconds to 4 minutes, and densitometry analysis using Quantity One software (Bio-Rad, Hercules, Calif.), was performed to determine the relative amount of IVTT expressed protein. Calculations were based on nM levels of the 6×His epitope on recombinant VirB9, which ranged from 1.6 to 206 nM for 0.015 to 2 μg protein.

### Binding of Recombinant Epitope-Tagged or IVTT-Expressed Proteins to Protein G-Coupled Carboxylate Beads via Anti-His or Anti-FLAG mAb

Recombinant VirB9 and negative-control RAP-1CT (Lopez et al., 2007) or IVTT expressed VirB9, OMP7, OMP8, and OMP9, were bound to 1 μm diameter protein G-conjugated carboxylate microspheres (Polysciences, Inc., Warrington, Pa.). One hundred μl of beads were washed 3 times with 750 μl of 0.1M Tris-HCL, 0.15M NaCl pH 7.4 (A/G buffer), with beads pelleted at 10,000×g for 5 minutes after each wash. Beads were then resuspended in 400 μl A/G buffer, containing 81.0 μg/ml of anti-FLAG mAb (Sigma-Aldrich) and incubated on a tumbler for 2 hours at 4° C., washed 3 times with A/G buffer, and resuspended in 400 μl of A/G buffer. Eleven μg recombinant VirB9 or RAP-1CT, or 20 μl of the IVTT reaction mixtures were added to 46.0 μl anti-FLAG-protein G-beads and incubated in a final volume of 200 μl on a tumbler for at least 2.5 hours at 4° C. The beads were washed 3 times in A/G buffer and resuspended in complete RPMI-1640 medium to yield a final protein G concentration of 10 μg/ml.

For screening the 50 candidate vaccine antigens, IVTT expressed proteins were bound to protein G-beads with penta-His-specific mAb (QIAGEN®). Lyophilized penta-His-specific mAb was rehydrated in A/G buffer, mixed with 10 μl of each IVTT expressed protein in a total volume of 500 μl, to yield a final concentration of the penta-His-specific mAb of 5 μg/ml, then incubated on a tumbler at 4° C. for 2 hours. Fifty μl protein G beads were added to each anti-penta-His mAb/IVTT reaction mixture. Samples were incubated on a tumbler 2 hours at 4° C., and then washed 3 times with 750 μl of A/G buffer, pelleting the beads at 16,000×g for 5 minutes after each wash. After the final wash, beads were resuspended in 400 μl of complete RPMI-1640 medium, to yield a final protein G concentration of 15.8 μg/ml.

A. marginale Outer-Membrane Specific T-Cell Lines

Peripheral blood mononuclear cells (PBMC) from outer-membrane immunized animals 04B90 and 04B91 (Lopez et al., 2005) were obtained 3 years after the final immunization to establish short-term T-cell lines. PBMC were cultured at 4×10⁶ cells per well in 24-well plates in a final volume of 1.5 ml of complete RPMI-1640 medium with 3 μg/ml of St. Maries A. marginale outer-membrane antigen (Lopez et al., 2005). After 7 days of incubation at 37° C. in 5% CO₂ in air, lymphocytes were washed and subcultured without antigen to a density of 7×10⁵ cells/well and 2×10⁶ fresh autologous irradiated (3,000 Rads) PBMC/well, serving as a source of APCs. Lymphocytes were incubated for 7 days and the “rested” T-cells then assayed for antigen-specific proliferation. Similar responses of short-term cell were previously found in cell lines from these animals to A. marginale antigens whether or not the cells were first enriched for CD4⁺ T-cells prior to setting up the cell lines (Lopez et al., 2007). Therefore, cell lines were used without first enriching for CD4⁺ T-cells.

### Lymphocyte Proliferation Assays

Prior to screening the large panel of IVTT expressed proteins, lymphocyte-proliferation assays were optimized with recombinant A. marginale VirB9 thioredoxin-FLAG-fusion protein bound to protein G-beads via anti-FLAG mAb. VirB9 has previously been shown to induce a CD4⁺ T-lymphocyte proliferative response in the outer-membrane immunized animals used in this study (Lopez et al., 2007). Recombinant A. marginale VirB9 and negative-control RAP-1CT coupled to protein G-beads via anti-FLAG mAb and unbound (empty) protein G-beads were assayed with T-cells and APC from immune animal 04B91 using bead dilutions determined as 0.04 to 5 μg/ml of protein G. One and 10 μg/ml recombinant VirB9 and RAP1-CT proteins were also tested. In a separate trial, IVTT-expressed VirB9, OMP7, OMP8, and OMP9 proteins coupled to the beads via anti-FLAG mAb were tested as above, and negative-controls included unbound (empty) protein G-beads and negative IVTT reaction mixture added to the protein G-beads with anti-FLAG mAb. Bead-bound VirB9 and the negative-control IVTT reaction mixture were also tested with a T-cell line from animal 4919 that had been immunized four times with a recombinant MSP1a-MSP1b fusion protein. A short term T-cell line stimulated with outer-membranes, rested, and tested with IVTT proteins as described above. Positive-control antigens were 10 μg/ml A. marginale outer-membranes and native MSP1 protein (Brown et al., Infect. Immun. 69, 6853, 2001a), and the negative-control antigen was 10 μg/m1 uninfected erythrocyte membranes (URBC).

For screening 50 IVTT expressed proteins, bead-bound proteins were tested with T-cells from immune animals 04B90 and 04B91 using bead dilutions determined as 1 and 5 μg/ml of protein G. Selected IVTT reaction mixtures that contained immunostimulatory antigens were also tested directly in the proliferation assay using 0.04, 0.2, and 1 μl of IVTT reaction mixtures. These amounts correspond to 0.2, 1 and 5 μg/ml of protein G-bead-bound antigen, assuming all IVTT expressed protein bound to the beads. For testing recombinant proteins, 1 and 10 μg/ml of protein was used. Positive-control antigens were 1 μg/ml of outer-membranes (Lopez et al., 2005) and 10 μg/m1 of native major-surface protein (MSP)2 (Brown et al., 2001a) purified from the St. Maries strain of A. marginale isolated from infected bovine blood.

As an additional control for the lack of mitogenicity of IVTT reaction products, selected IVTT-expressed bead-bound proteins were tested on a short-term cell line from Babesia bovis infected animal C15. PBMC were cultured for one week with 5 μg/ml B. bovis merozoite membranes (CM) and then cells were cryopreserved in liquid nitrogen. Thawed cells were washed and cultured with irradiated C15 PBMC as APCs, and 1 and 5 μg/ml protein G-bead bound IVTT-expressed amino peptidase, VirB9, OMP9, Ana29, elongation factor-tu (EF-Tu), and OMP4. These were selected because they induced significant proliferation of T-cells from outer-membrane vaccinates.

For all proliferation assays, short-term T-cell lines were incubated with antigens at a density of 3×10⁴ T-cells/well with 2×10⁵ irradiated autologous PBMC as a source of APC/well in triplicate 100 μl cultures. Cells were cultured for 4 days, and radiolabeled during the last 18 hours with 0.25 μCi of [³H] thymidine (New England Nuclear, Boston, Mass.), harvested onto glass filters. Radionucleotide incorporation was measured with a Beta-plate 1205 liquid scintillation counter (Wallac, Turku, Finland). Results are expressed as either mean counts per minute (CPM) +1 SD or the stimulation index (SI), determined by dividing the mean CPM of replicate cultures with antigen by the mean CPM of triplicate cultures with a negative-control antigen. The NCSS 2001 statistical software package was used to determine significant differences in lymphocyte proliferation. The assumption of normality was assessed by skewness, kurtosis and the omnibus normality of residuals. Samples were compared using one way ANOVA and the Fisher's Least Significant Difference test with an experimental alpha value=0.05. A. marginale outer-membranes and native MSP2 were compared to negative-control uninfected bovine erythrocyte membranes, IVTT-expressed proteins were compared with either IVTT-expressed OMP2 or OMP3, depending on which protein was included in the assay, and recombinant proteins expressed in E. coli were compared to B. bovis RAP1-CT or the C-terminal half of OMP10, expressed in the same vector as thioredoxin fusion proteins, and shown not to elicit specific proliferation.

### Western Blotting of Selected, Bead-Purified IVTT-Expressed Proteins

IVTT expressed amino peptidase, VirB9, OMP9, Ana29, EF-Tu, and OMP4 were subjected to SDS-PAGE and immunoblotting to verify the predicted molecular mass of each protein. IVTT reaction mixtures were affinity-purified on anti-His mAb-beads as described, except that the unbound IVTT reaction mixture was saved after the beads were pelleted. Twenty-four μl of washed beads to which IVTT-expressed proteins were bound or supernatant obtained after pelleting the beads before washing were solubilized in 6 μl 5×SDS-PAGE sample buffer, and loaded onto a 4-20% gradient gel (Bio-Rad). Following electrophoresis, proteins were transferred to a nitrocellulose membrane and incubated for 1 hour at room temperature with I-Block containing 0.5% Tween-20. The membrane was then incubated 1 hour at room temperature with alkaline phosphatase labeled anti-FLAG mAb (Sigma-Aldrich) diluted 1:2,000 in I-Block and washed twice in washing buffer. Chemiluminescent detection was performed with the WESTERN-STAR™ Immunodetection system (Applied Biosystems).

### Results and Discussion

Holstein cattle expressing distinct MHC class II haplotypes were immunized three years prior to the study disclosed herein with a protective A. marginale outer-membrane fraction, which induces multiple T-cell and antibody specificities. This model enabled the evaluation of known and putative T-cell antigens using the disclosed method and polyclonal short-term T-cell lines derived from the outer-membrane vaccines.

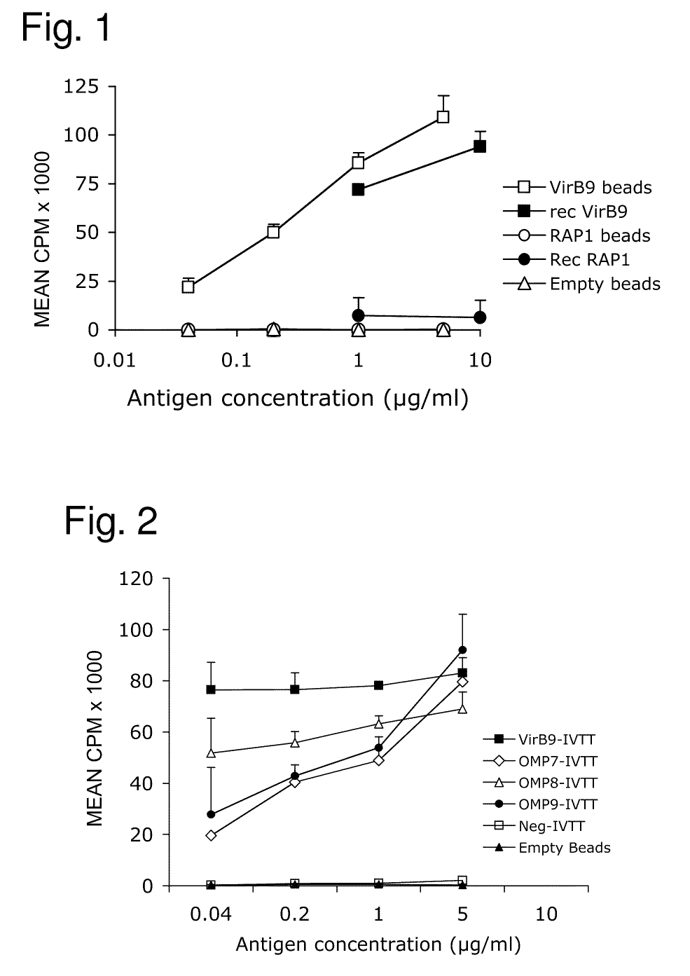

First, optimal protein G-bead concentrations were determined. A known immunostimulatory CD4⁺ T-cell antigen, VirB9, was expressed as a FLAG epitope-tagged recombinant protein in E. coli and bound to protein G-beads via anti-FLAG mAb. Bead-bound VirB9, but not bead-bound negative-control RAP1-CT from B. bovis, stimulated dose-dependent proliferation of immune T-cells using dilutions of beads based on protein G concentrations of 0.04-5 μg/ml (FIG. 1). As expected, recombinant VirB9 induced significant responses when compared with RAP-1CT.

It was next determined if protein expressed by IVTT could elicit recall T-lymphocyte proliferative responses from a polyclonal lymphocyte population. PCR was performed on genes encoding VirB9 and OMP7, which induce IgG2 responses in the outer-membrane vaccines, and related OMP8 and OMP9, to generate gene-specific TAP fragments. TAP fragments encoding C-terminal FLAG and 6×His epitopes produced proteins as detected by dot-blot analysis using FLAG-specific mAb. IVTT-expressed proteins were then bound to protein G-beads via anti-FLAG mAb, and tested in T-lymphocyte proliferation assays. All four bead-bound IVTT-expressed proteins induced statistically significant proliferation of T-lymphocytes from outer-membrane vaccine 04B91 when compared to negative-control IVTT reaction mixture and empty beads (FIG. 2). Although the negative-control IVTT reaction mixture did not have any mitogenic activity, IVTT-expressed VirB9 and the negative-control IVTT reaction mixture was also tested for mitogenic activity using a short-term T-cell line established from a calf immunized with a recombinant MSP1 fusion protein consisting of MSP1a T-cell epitopes and MSP1b 1. When compared with the negative-control erythrocyte membrane antigen, T-cells from this animal had significant responses to A. marginale outer-membranes and native MSP1 protein, but not to any concentration of either IVTT reaction product (FIG. 3), ruling out non-specific activity of the IVTT reaction products. A large-scale screening of 50 IVTT-expressed vaccine candidates was then performed using T-cells from the two outer-membrane vaccines. These antigens were selected for IVTT expression based on predicted bacterial surface location and genome annotation and included several known T-cell antigens. OMP2 and OMP3 were included as negative-control antigens because they are not expressed by A. marginale in erythrocytes, and therefore, not present in the outer-membrane immunogen. PCR was performed to generate gene-specific TAP fragments encoding an N-terminal 6×His epitope and C-terminal FLAG epitope. Expression of protein from individual TAP fragments was confirmed by dot-blot analysis using penta-His-specific mAb. The relative nM level of 6×His expressed in 1 μl of each IVTT product was determined from a standard curve of two-fold dilutions of recombinant his-tagged VirB9. Based on densitometry, the relative molarities of the expressed A. marginale proteins ranged from 1.6 to 203.6 nM 6×His (Table 2).

TABLE 2
Antigens identified by screening IVTT products with CD4⁺ T-cells

| | Proliferation (Stimulation Index)^{a} | | |
|---|---|---|---|
| | Animal Number | | IVTT Expression^{b} |
| Protein | #04B90 | #04B91 | (nM 6xHis) |
| Positive-control antigens | | | |
| A. marginale outer-membranes | 7.8 | 130.8 | N/A^{c} |
| Native MSP2 | 2.8 | 140.0 | N/A |
| IVTT-expressed proteins | | | |
| Known antigens | | | |
| MSP2 | 3.2 | 380.8 | 34.1 +/− 0.6 |
| MSP3 | 2.1 | 78.5 | 9.6 +/− 0.6 |
| MSP4 | 2.5^{d} | 10.2 | 6.3 +/− 0.6 |
| VirB9 | 1.8 | 178.5 | 33.5 +/− 4.9 |
| VirB10 | 2.2 | 29.2^{d} | 121.6 +/− 7.0 |
| Newly tested antigens | | | |
| OMP1 | 2.2^{d} | 6.5 | 108.6 +/− 14.5 |
| OMP2 | 1.0 | 1.0 | 1.6 +/− 0.2 |
| OMP3 | 1.0 | 1.0 | 8.0 +/− 1.1 |
| OMP4 | 2.8 | 64.7 | 31.8 +/− 8.9 |
| OMP8 | 1.1 | 93.7 | 8.2 +/− 0.8 |
| OMP9 | 1.7^{d} | 380.1 | 35.8 +/− 1.7 |
| OpAg3 | 1.6^{e} | 0.6 | 3.8 +/− 0.4 |
| EF-Tu | 1.9 | 128.6 | 3.5 +/− 0.4 |
| Ana29 | 1.8 | 16.3 | 34.6 +/− 6.4 |
| OrfX (VirB2) | 1.6 | 0.6 | 203.6 +/− 0 |
| Patatin | 1.5^{d} | 0.6 | 70.3 +/− 7.5 |
| Amino peptidase (pepA) | 3.3 | 1.0 | 68.0 +/− 4.3 |
| OMA87 | 1.5^{e} | 27.1 | 78.7 +/− 8.4 |
| AAAP AM879 | 2.8 | 1.0 | 33.5 +/− 4.9 |
| AAAP AM880 | 3.0 | 0.9 | 22.2 +/− 4.7 |
| Peptidoglycan lipoprotein | 1.5^{d} | 2.4 | 11.6 +/− 0.8 |
| Hyp. protein AM197 | 1.6^{d} | 0.4 | 44.2 +/− 0.8 |
| Hyp. protein AM366-2 | 2.6 | 0.8 | 8.2 +/− 0.8 |
| Hyp. protein AM529 | 1.4^{e} | 1.1 | 10.3 +/− 0.3 |
| Family putative OMPs | 2.9^{d} | 1.1 | 39.1 +/− 0.1 |
| AM779 | | | |

^{a}Short-term T-cell lines from two animals immunized with A. marginale outer-membranes were tested in proliferation assays using 1 or 5 μg/ml protein G-beads, 1 μg/ml A. marginale outer-membranes, or 10 μg/ml native MSP2. Fifty IVTT-expressed proteins were tested, and only those that stimulated a significant response are shown. Stimulation indices were derived by dividing the mean CPM of triplicate cultures of cells with antigen by the mean CPM of triplicate cultures containing negative-control antigens. For IVTT reactions, these were either OMP2- or OMP3-beads, and for outer-membranes and native MSP2, this was uninfected erythrocyte membranes. Results are presented as the stimulation index using 5 μg/ml protein G-beads, unless indicated otherwise. Those in bold-faced type were significantly different from negative-control antigen at both antigen concentrations (P < 0.05), unless noted otherwise. The NCSS 2001 statistical software package was used. Samples were compared using one way ANOVA and the Fisher's Least Significant Difference test, where σ = 0.05. Animal #04B90 had high background CPM, resulting in relatively low stimulation indices.
^{b}The relative level of protein expressed in the IVTT reaction was determined by comparison with a standard curve of recombinant E. coli-expressed VirB9, which contains 103 nM 6xHis/μg, on dot blots using 0.015-2.0 μg VirB9 and 1 μl IVTT reaction. Blots were exposed for 15, 30, 60, 120, and 240 seconds and the spots were analyzed by densitometry. The results are presented as the mean nM 6xHis +/− 1 SD of two exposure times that fell within the linear portion of the standard curve.
^{c}Not applicable
^{d}Only 5 μg/ml protein G-bead-bound antigen was stimulatory.
^{e}Only 1 μg/ml protein G-bead-bound antigen was stimulatory.

IVTT-expressed proteins were then purified by binding to protein G-beads via anti-His mAb and tested for T-lymphocyte stimulation. Of the 50 IVTT-expressed proteins, 23 stimulated significant proliferative responses of T-cells from one or both animals. These included MSP2, MSP3, MSP4, VirB9, and VirB10, which were previously shown to induce CD4⁺ T-lymphocyte responses in outer-membrane vaccines and the newly described OMP8 and OMP9 (FIG. 2). Only those proteins stimulating significant responses are shown in Table 2. The relatively lower stimulation indices reported for animal 04B90 resulted from very high background proliferation to medium typically observed for this animal. Importantly, screening of IVTT-expressed antigens identified an additional five proteins as immunogenic for T-cells from both animals. These are OMP4, OMP9, EF-Tu, OMA87, and Ana29 (Table 2).

A significant lymphocyte response to OMP8 was again obtained from animal 04B91, and significant responses to MSP4 and 12 new T-cell antigens were obtained from animal 04B90. Of these new antigens, appendage-associated proteins (AAAPs) were also recognized on 2-D immunoblots with immune serum IgG2 from animal 04B90, consistent with T-cell recognition. Although statistically significant, many of these responses from animal 04B90 were relatively low (due to high background proliferation), or detected using only one antigen concentration. Thus, proliferation assays were repeated for eight antigens (OMP1, OMP9, OpAg3, EF-Tu, patatin, peptidoglycan lipoprotein, and hypothetical proteins AM197 and AM529). In each case, a significant response was again obtained, demonstrating the reproducibility of this method of screening the bead-bound IVTT expressed proteins for specific T-cell recognition.

The finding that 27 IVTT-expressed proteins were not stimulatory rules out non-specific mitogenic activity for those antigens that did stimulate significant proliferation, as all proteins were expressed, purified, and tested under identical conditions. Nevertheless, six IVTT-expressed proteins (amino peptidase, VirB9, OMP9, Ana29, EF-Tu, and OMP4) that stimulated T-cell responses from outer-membrane immunized animals were also tested on a short-term cell line established from a Babesia bovis infected cow. As demonstrated in FIG. 4, T-lymphocytes responded to B. bovis merozoite membrane antigen, but not to any of the IVTT-expressed proteins from A. marginale. This result definitively rules out any non-specific mitogenic activity of these antigens.

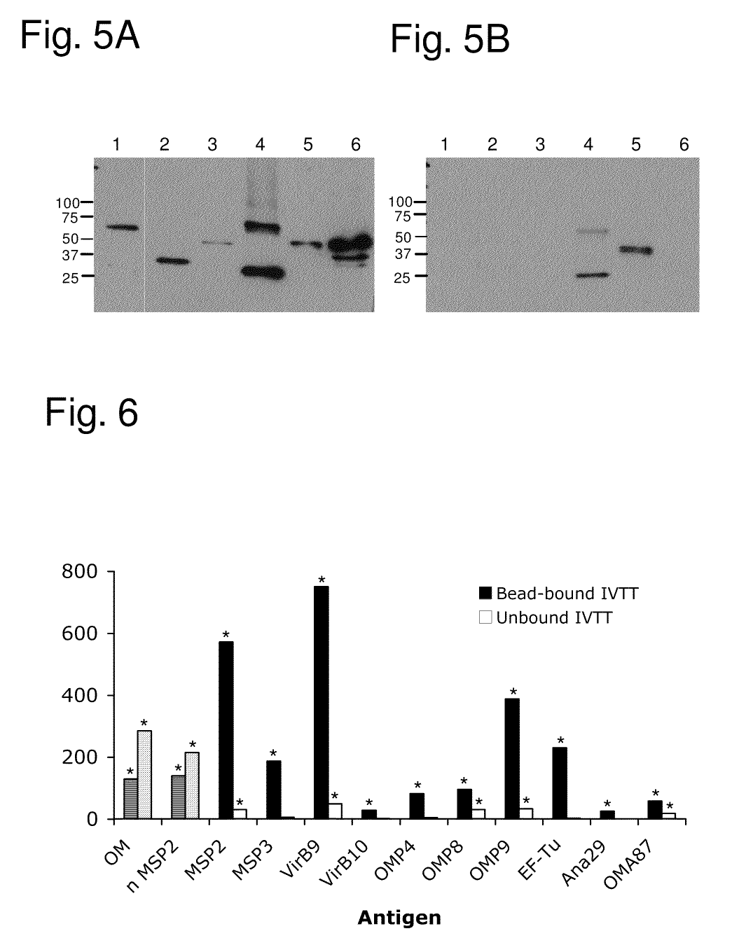

To verify that the expressed proteins are of the predicted size, amino peptidase, VirB9, OMP9, Ana29, EF-Tu and OMP4 were analyzed by Western blotting using anti-FLAG mAb. Antigen affinity purified on the beads (FIG. 5A) was compared with IVTT reaction mixture that remained after incubating the anti-His mAb-protein-G coupled beads with the IVTT reaction mixture (FIG. 5B). First, all six proteins migrated at the correct estimated molecular weight which includes ˜1.8 kDa for the 6×His and FLAG epitopes. These are: amino peptidase (lane 1, 59 kDa), VirB9 (lane 2, 33 kDa), OMP9 (lane 3, 45 kDa), Ana29 (lane 4, 30 kDa), EF-Tu (lane 5, 45 kDa), and OMP4 (lane 6, 46 kDa). Second, this test shows that amino peptidase, VirB9, OMP9, and OMP4 were only detected as bead-bound antigens, whereas Ana29 and EF-Tu were detected both as bead-bound antigens and in the supernatant remaining after bead-affinity purification. Thus, in four of six reaction mixtures, the proteins appear to be completely bound by the affinity purification. Third, Ana29 appears to form a dimer as both the predicted 30 kDa band and a band of ˜60 kDa are detected with the anti-FLAG mAb.

Significant CD4⁺ T-cell proliferation from animals 04B90 and 04B91 to E. coli-expressed recombinant VirB9 and VirB10 were shown previously (Lopez et al., 2007). However, to validate the IVTT screening assay additional recombinant proteins were tested. Four immunostimulatory proteins (EF-Tu, OMPs 7, 8, and 9) and six non-stimulatory proteins (OMPs 2, 3, 10, 11, 12, and 14) were conventionally expressed in E. coli, and recombinant proteins were tested in T-lymphocyte proliferation assays (Table 3). Overall, there was strong concordance between IVTT-expressed and conventionally expressed recombinant A. marginale proteins to stimulate specific T-lymphocyte proliferation. Recombinant OMP7, OMP9, and EF-Tu induced significant proliferative responses in T-lymphocytes from both outer-membrane vaccines. In contrast, IVTT expressed proteins that did not stimulate specific T-lymphocyte proliferation also failed to stimulate responses as E. coli-expressed recombinant proteins (Table 3). Recombinant OMP8 reproducibly stimulated T-cells from animal 04B91, but the results for animal 04B90 were inconsistent, with some preparations of antigen being stimulatory and others not (Table 2). It was observed that recombinant proteins, if not completely free of contaminating E. coli proteins and detergents used in purification, can either give false-positive or false-negative results in lymphocyte-proliferation assays. Therefore, high-throughput screening of antigens expressed by IVTT using the disclosed methods is surprisingly as sensitive as and more specific than using recombinant proteins obtained from an E. coli source.

TABLE 3
Lymphocyte proliferation to E. coli-expressed proteins

| | Proliferation (Stimulation Index)^{a} | | | |
|---|---|---|---|---|
| | Animal 04B90 | | Animal 04B91 | |
| Protein | 1 μg/ml | 10 μg/ml | 1 μg/ml | 10 μg/ml |
| Expt. 1 | | | | |
| OMP10 | 0.1 | 0.1 | 0.8 | 0.6 |
| OMP11 | 0.1 | 0.1 | 0.8 | 0.6 |
| OMP14 | 0.1 | 0.1 | 15.1 | 0.7 |
| A. marginale OM | 13.5 | 13.3 | 195.1 | 1,047.2 |
| Expt. 2 | | | | |
| EF-Tu | 2.7 | 3.9 | 56.2 | 49.2 |
| A. marginale OM | 4.8 | 6.6 | 135.0 | 247.7 |
| Expt. 3 | | | | |
| OMP2 | 1.1 | 1.1 | 1.1 | 0.4 |
| OMP3 | 0.7 | 0.3 | 1.3 | 0.3 |
| OMP7 | 2.6 | 6.0 | 36.4 | 37.8 |
| OMP8 | 1.5 | 1.3 | 48.0 | 43.7 |
| OMP9 | 2.5 | 10.0 | 92.3 | 62.7 |
| OMP12 | 0.9 | 0.5 | 1.5 | 0.4 |
| A. marginale OM | 7.8 | 3.5 | 23.2 | 41.8 |

^{a}Short-term T-cell lines from two animals immunized with outer-membranes were tested in proliferation assays using 1 and 10 μg/ml recombinant protein expressed in E. coli or A. marginale outer-membranes as a positive-control. Stimulation indices (SI) were derived by dividing the mean CPM of replicate cultures with antigen by the mean CPM of replicate cultures containing negative-control antigen consisting of the B. bovis RAP1-CT or the C-terminal half of OMP10 expressed in the same vector (animal 04B90, Expt. 3), or for A. marginale outer-membranes, membranes from uninfected erythrocytes. Samples were compared using one way ANOVA and the Fisher's Least Significant Difference test, where σ = 0.05. Results in bold-faced type are significantly greater than control antigen.

To determine whether IVTT-expressed antigens not subjected to bead-affinity purification could induce specific T-lymphocyte proliferation, polyclonal T-cell lines from the high-responder animal 04B91 were tested with 0.04, 0.2, and 1 μl of selected IVTT reaction mixtures. For VirB9 and VirB10, we also tested 0.008 and 5 μl of reaction mixture. IVTT reaction mixtures of 0.2 and 1 μl correspond to approximately 1 and 5 μl, respectively, of IVTT-expressed antigen coupled to protein-G beads, assuming all antigen bound. Significant T-lymphocyte stimulation was achieved with only half of the IVTT reaction mixtures, and for these the levels of stimulation were dramatically lower when compared to responses to protein G-bead bound antigen (FIG. 6). These poor responses were not due to a generally poor T-lymphocyte response to antigen, as the responses to A. marginale outer-membranes and native MSP2 were higher in the assay testing the IVTT reaction mixtures (FIG. 6). For all antigens, higher responses were obtained with 0.04 μl than with 0.2 μl of IVTT reaction mixtures (FIGS. 4), and 5 μl of VirB9 and VirB10 IVTT reaction mixtures did not induce proliferation. These results suggest that the IVTT reaction mixture inhibits antigen-driven T-cell proliferation at the higher concentrations. When 0.008 μl of IVTT products were tested, there was stronger proliferation with VirB9 (SI=139.3) but no improvement with VirB10 (SI=2.2). Thus, enriching the IVTT-expressed proteins by binding to protein G-beads increased the sensitivity of the T-cell proliferation assay by affinity purifying the antigen.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

We claim:

1. An in vitro method for high-throughput identification of antigens from a pathogen of interest, comprising:
generating a transcriptionally active PCR (TAP) product of an open reading frame of an antigen candidate of a pathogen of interest, wherein the TAP product comprises an identifiable tag, a transcription promoter and a transcription terminator;
expressing the TAP product in an in vitro translation transcription (IVTT) system to generate a candidate vaccine antigen;
purifying the candidate vaccine antigen by contacting the candidate vaccine antigen with an antibody that specifically binds the identifiable tag, wherein the antibody that specifically binds the identifiable tag is attached to a microbead.
contacting the purified candidate vaccine antigen to isolated antigen presenting cells (APCs);
contacting the APCs with isolated T-cells; and
detecting antigen induced activation of the T-cells, wherein activation of the T-cells identifies the candidate vaccine antigen as an antigen.

2. The method of claim 1, wherein generating a TAP product comprises:
contacting the open reading frame of the antigen candidate with a first oligonucleotide primer and second oligonucleotide primer capable of hybridizing to and amplifying the open reading frame, wherein the first oligonucleotide primer comprises a nucleotide sequence capable of hybridizing to the open reading frame and a first nucleic acid sequence heterologous to the open reading frame, and wherein the second oligonucleotide primer comprises a nucleotide sequence capable of hybridizing to the open reading frame, a second nucleic acid sequence heterologous to the open reading frame and a nucleic acid sequence encoding the identifiable tag;
amplifying the open reading frame, thereby generating a first amplification product;
contacting the first amplification product with a third oligonucleotide primer and fourth oligonucleotide primer capable of hybridizing to and amplifying the first amplification product, wherein the third oligonucleotide primer comprises a nucleic acid sequence encoding the transcription promoter and wherein the fourth oligonucleotide comprises a nucleic acid sequence encoding the transcription terminator; and
amplifying the first amplification product, thereby generating the TAP product comprising the identifiable tag, the transcription promoter and the transcription terminator.

3. The method of claim 1, wherein the identifiable tag comprises a FLAG tag.

4. The method of claim 1, wherein the identifiable tag comprises a 6×His tag.

5. The method of claim 1, wherein the identifiable tag comprises a FLAG tag and a 6×His tag.

6. The method of claim 1, wherein the transcription promoter comprises a T7 promoter,

7. The method of claim 1, wherein the transcription terminator comprises a T7 terminator.

8. The method of claim 1, wherein the IVTT system is an Escherichia coli based IVTT system.

9. The method of claim 2, wherein the first oligonuceotide primer further comprises a second identifiable tag.

10. The method of claim 1, wherein the APCs comprise B-cells, dendritic cells, macrophages or a combination thereof.

11. The method of claim 1, wherein the APCs are obtained from a subject that has not been immunized with one or more antigens from the pathogen of interest.

12. The method of claim 1, wherein the T-cells comprise CD4⁺ T-cells.

13. The method of claim 1, wherein the T-cells comprise CD8⁺ T-cells.

14. The method of claim 1, wherein the T-cells are obtain from a subject previous immunized with one or more antigens from the pathogen of interest.

15. The method of claim 1, wherein detecting antigen induced activation of the T-cells comprises detecting one or more of proliferation of the T-cells, cytokine secretion, cytokine secreting cell enumeration, or cell surface activation molecule detection.

16. The method of claim 1, wherein the pathogen of interest is a bacterial, viral, fungal or parasitic pathogen of interest.

17. The method of claim 16, wherein the pathogen of interest is A. marginale.

18. The method of claim 1, comprising generating TAP products of multiple antigen candidates.

19. The method of claim 18, wherein the TAP products of multiple antigen candidates are generated from a genome sequence of the pathogen of interest.

20. The method of claim 19, wherein the genome sequence is the genome sequence of A. marginale.

21. An immunogenic composition comprising an antigen identified using the method of claim 1.

22. An immunogenic composition comprising one or more of a OMP1, OMP2, OMP3, OMP4, OMP8, OMP9, OpAg3, EF-Tu, Ana29, VirB2, Patatin, Amino peptidase (pepA), OMA87, AAAP AM879, AAAP AM880, Peptidoglycan lipoprotein, AM197, AM366-2, AM529 or AM779 polypeptide from A. marginale or an immunogenic fragment thereof or a nucleic acid encoding the polypeptide.

23. A method of treating and/or inhibiting an infection by A. marginale, comprising:
selecting a subject for treatment that has, or is at risk for developing, an infection by A. marginale;
administering to the subject a therapeutically effective amount of the immunogenic composition of claim 22, thereby treating and/or inhibiting an infection by A. marginale.
